(19) United States
(12) Patent Application Publication
(10) Pub. No.: US 2023/0285529 A1
(43) Pub. Date: Sep. 14, 2023

(54) IMMUNOSTIMULATORY COMPOSITIONS COMPRISING SOLUBLE PARASITE EXTRACTS AND USES THEREOF

(71) Applicant: GRIFFITH UNIVERSITY, Nathan, Queensland (AU)

(72) Inventors: Michael GOOD, Nathan, Queensland (AU); Danielle STANISIC, Nathan, Queensland (AU); Ashwini Kumar GIDDAM, Nathan, Queensland (AU); Aloysious SSEMEGANDA, Nathan, Queensland (AU)

(73) Assignee: GRIFFITH UNIVERSITY, Nathan, Queensland (AU)

(21) Appl. No.: 17/778,739

(22) PCT Filed: Nov. 23, 2020
(86) PCT No.: PCT/AU2020/051269

§ 371 Date: May 20, 2022

(30) Foreign Application Priority Data
Nov. 21, 2019 (AU) 2019904410

(51) Int. Cl. A61K 39/015; A61P 33/02; A61K 39/018

(52) U.S. Cl. CPC A61K 39/015; A61P 33/02; A61K 39/018; A61K 2039/55555

(57) ABSTRACT

Disclosed are compositions for stimulating a protective or therapeutic immune response to an apicomplexan parasite such as those from the Plasmodium or Babesia genus. More particularly, the compositions comprise a soluble parasite extract. The extract may be free of red blood cell components and/or contained in or associated with a particle such as a liposome. The compositions and methods disclosed herein are particularly useful in the prevention and treatment of parasitic diseases.

### RELATED APPLICATIONS

This application is a 35 U.S.C. § 371 National Phase Entry Application of International Patent Application No. PCT/AU2020/051269 filed on Nov. 23, 2020, which designated the U.S., and which claims benefit under 35 U.S.C. § 119 of Australian Provisional Application No. 2019904410 entitled “Immunostimulatory Compositions and Use Thereof” filed 21 Nov. 2019, the contents of which are incorporated herein by reference in their entireties.

### FIELD OF THE INVENTION

This invention relates generally to compositions and methods for stimulating immune responses. More particularly, the present invention relates to the use of parasite preparations that stimulate an immune response to a parasite antigen in compositions and methods for stimulating protective or therapeutic immune responses to a parasite. The compositions and methods of the present invention are particularly useful in the prevention and treatment of parasitic diseases.

### BACKGROUND OF THE INVENTION

Malaria vaccine development has focused primarily on the subunit approach in which a restricted number of parasite antigens are formulated with delivery systems and adjuvants to enhance immune responses against specific antigenic epitopes. However, this strategy has shown limited success in field studies with the vaccines being only moderately efficacious; furthermore, immunity is short-lived (see, Genton et al., 2002; Sirima et al., 2011; Ewer et al., 2013; and Olotu et al., 2016). This can be attributed to poor immunogenicity and failure to maintain parasite-specific antibody responses as well as the diversity of the antigens used in these vaccines (see, Ogutu et al., 2009; Thera et al., 2011; and Neafsey et al., 2015).

These shortcomings have led to renewed interest in whole parasite (WP) vaccines. Substantial progress with this strategy has been made with irradiated sporozoites (PfSPZ) (see, Seder et al., 2013; Ishizuka et al., 2016; Epstein et al., 2017; and Lyke et al., 2017) and chemo-attenuated sporozoite infections (PfSPZ-cVac) (see, Mordmuller et al., 2017), which have demonstrated significant efficacy in controlled human malaria infection studies (CHMI) as well as in natural infection studies (see, Sissoko et al., 2017). Less progress has been made with WP blood-stage malaria vaccines; however genetically modified WP vaccines (see, Ting et al., 2008) and chemically attenuated WP vaccines (see, Good et al., 2013; and Raja et al., 2016) have been tested in murine models, and the latter in clinical studies (see, Stanisic et al., 2018) where they were shown to induce potent immune responses in malaria naïve volunteers. Nevertheless, there are significant challenges regarding storage, cold-chain maintenance and delivery of WP blood-stage vaccines.

An alternative approach is to use inactivated WP antigens, which have been adjuvanted (see, Su et al., 2003; and Pinzon-Charry et al., 2010). Unlike live-attenuated vaccines, which have the ability to naturally deliver whole antigens and initiate robust immune responses, inactivated antigen-based vaccines require potent adjuvants and delivery systems. Progress with inactivated WP vaccines in human studies has been restricted due to the limited number of suitable human-compatible adjuvants (see, Coler et al., 2009; and Brito & O'hagan, 2014). Despite this, liposomes are becoming an increasingly attractive adjuvant and antigen delivery platform for the development of inactivated WP vaccines and have been used clinically for the RTS, S subunit vaccine (see, Olotu et al., 2016; and Brito & O'hagan, 2014).

The physicochemical properties of liposomes can be modified to achieve the desired immune responses and adjuvant properties (see, Perrie et al., 2016; and Ssemaganda et al., 2019). Mannosylation of liposomes is known to increase the targeting of cargo antigens to professional APCs and induce potent antibody and cell-mediated immune responses (see, Copland et al., 2003; and Vyas et al., 2010). We recently demonstrated that mannosylated liposomes encapsulating whole rodent malaria parasites (P. chabaudi and P. yoelii) protected mice from homologous challenge infection (see, Giddam et al., 2016).

Induction of alloantibodies to red blood cell antigens following inoculation of WP vaccines containing red cell membranes still remains a major challenge and could lead to transfusion reactions and autoimmune hemolytic anaemia. This remains an issue even if the parasites are grown in blood type O, Rh Neg (‘universal donor’) blood (see, Stanisic et al., 2018).

### SUMMARY OF THE INVENTION

The present inventors have surprising discovered that a soluble parasite extract can be used to elicit or enhance an immune response to a parasite in a subject. This administration of a soluble parasite extract has particular advantages in the prevention or treatment of parasitic diseases.

Thus, in one aspect, the present invention provides immunostimulatory compositions for stimulating an immune response to a parasite in a subject. The immunostimulatory compositions of the invention generally comprise a soluble parasite extract, wherein the parasite extract is substantially free of insoluble parasite components or RBC components.

Typically, wherein the soluble parasite extract is contained in or otherwise associated with a particle. In some embodiments of this type, the soluble parasite extract is completely free of insoluble parasite components or red blood cell (RBC) components.

In another aspect, the immunostimulatory compositions comprise a soluble parasite extract contained in or otherwise associated with a particle.

In some embodiments, the soluble parasite antigen component is at least partially purified from red blood cell components. In some particularly preferred embodiments, the soluble parasite antigen component is substantially free of red blood cell components.

In some embodiments, the soluble parasite extract comprises, consists, or consists essentially of substantially all the soluble parasite molecules present in the parasite.

In some of the same embodiments and some other embodiments, the soluble parasite extract is substantially free of detergent.

Typically, the parasite is an apicomplexan. Therefore in some embodiments, the apicomplexan parasite belongs to a genus selected from Plasmodium and Babesia. By way of an illustrative example, in some embodiments the Plasmodium parasite is selected from the species Plasmodium falciparum, P. malariae, P. ovale, P. vivax, and P. knowlesi, or a combination thereof. In some alternative embodiments, the Babesia parasite is selected from the species Babesia bigemina, B. bovis, B. caballi, B. canis, B. divergens, B. microti, and B. motasi, or a combination thereof. In some embodiments, the composition comprises two or more species of parasite from a single genus.

In some embodiments, wherein the particle is a lipid vesicle. Preferably, the particle is a liposome. The compositions of the invention may comprise particles that are capable of being phagocytosed by an immune cell.

In some embodiments, wherein the composition comprises a cell-targeting ligand. Typically, the cell-targeting ligand targets the particle to an immune cell (e.g., an antigen-presenting cell (APC)). By way of an illustrative example, the immune cell may be a dendritic cell and/or macrophage. In some exemplary embodiments, the cell-targeting ligand comprises a lipid anchor component, a linker component, and an oligosaccharide component. The oligosaccharide component may comprise at least one mannose residue, and preferably two, three or four mannose residues. In some embodiments, the lipid anchor component comprises one or two lipid molecules. For example, the lipid anchor component may comprise at least one palmitic acid molecule. In some of the same embodiments and in alternative embodiments, the linker component comprises at least one amino acid residue, and preferably two, three, or four amino acid residues. Preferably, the amino acid residues are comprised of lysine and serine amino acid residues. In some embodiments, the linker component comprises one or more polyethylene glycol (PEG) molecules.

In some of the same embodiments and some other embodiments, the composition further comprises an adjuvant. Suitably, the adjuvant is a TLR4 agonist. By way of an example, the TLR4 agonist may be a Monophosphoryl Lipid A (MPLA) molecule, or a derivative thereof.

In some embodiments, the adjuvant is encapsulated within the particle, presented on the surface of the particle, or located outside of the particle. In some specific embodiments, the particle may be a liposome, and the adjuvant is at least partially embedded in the lipid bilayer of the liposome.

Typically, the composition is formulated as a vaccine. In some embodiments, the composition is cryopreserved or freeze dried. In some preferred embodiment, the composition is lyophilized. In some alternative embodiments, the composition is rehydrated.

In another aspect, the invention provides methods of preparing immunomodulatory compositions for eliciting an immune response to a parasite antigen, the method comprising:
harvesting parasitized red blood cells (pRBCs);
lyse the pRBC cells under conditions sufficient to lyse the membrane of red blood cells but not sufficient to significantly lyse the parasite membranes;
harvesting the insoluble fraction of the pRBC lysate, wherein the insoluble fraction comprises red blood cell membranes and whole parasites;
lyse the parasite membranes; and
harvest the soluble parasite fraction, wherein the soluble parasite fraction comprises soluble parasite antigens;
to thereby produce an immunogenic composition sufficient to elicit an immune response to the parasite.

Typically, the pRBCs are lysed by exposing the cells to a chemical lysis buffer (e.g., saponin in PBS).

Typically, the step of harvesting the insoluble fraction (including parasites and RBC membranes) of the pRBC lysate is performed by centrifugation. In some embodiments, the step of harvesting the insoluble fraction of the pRBC lysate further comprises washing the insoluble fraction in buffer.

Typically, the step of lysing the parasite membrane comprises exposing the parasites to one or more freeze-thaw cycles, preferably at least three, at least four, or at least five freeze-thaw cycles.

In some of the same embodiments and some other embodiments, the step of lysing the parasite membrane comprises exposing the parasites to sonication.

Typically, the step of harvesting the soluble fraction comprises centrifugation. In some of the same embodiments and other embodiments, the step of harvesting the soluble fraction comprises filtration.

In some of the same embodiments and some other embodiments, the step of harvesting the soluble fraction further comprises the use of immune-affinity depletion to remove the red blood cell membranes. In some embodiments of this type, the immune-affinity depletion is performed using an anti-glycophorin A antibody.

In some embodiments, the step of harvesting the soluble fraction comprises two or more of: (i) immune-affinity depletion; (ii) centrifugation; and (iii) filtration.

In yet another aspect, the present invention provides methods of eliciting an immune response to a parasite antigen in a subject, the method comprising administering a composition comprising a soluble parasite extract contained in or otherwise associated with a particle, to thereby elicit an immune response in the subject.

In still yet another aspect, the present invention provides methods of preventing or treating a parasitic disease in a subject, the method comprising administering a composition comprising a soluble parasite extract contained in or otherwise associated with a particle, to thereby prevent or treat the parasitic disease in the subject.

Typically, the soluble parasite extract is substantially free of any red blood cell components.

Typically, the immune response comprises a T cell immune response (e.g., a CD4+ T cell response and/or a CD8+ T cell response).

In some embodiments, the composition is administered to the subject by subcutaneous injection, intramuscular injection, intravenous injection, or intraperitoneal injection.

In still yet another aspect, the present invention provides uses of soluble parasite extracts contained in or otherwise associated with a particle, in the manufacture of a medicament for enhancing or stimulating an immune response to a parasite antigen in a subject. Typically, the soluble parasite extracts are substantially free from RBC components.

In still yet another aspect, the present invention provides uses of soluble parasite extracts contained in or otherwise associated with a particle, in the manufacture of a medicament for the treatment or prophylaxis of a disease and/or condition associated with the presence of the parasite in a subject. Typically, the soluble parasite extracts are substantially free from RBC components.

In yet still another aspect, the present invention provides immunostimulatory compositions comprising a whole parasite extract contained in or otherwise associated with a particle, wherein the whole parasite antigen component is substantially free or completely free of red blood cell components.

In yet still another aspect, the present invention provides methods of preventing or treating malaria in a subject, the method comprising the method comprising administering a composition comprising a soluble Plasmodium parasite extract contained in or otherwise associated with a particle; to thereby prevent or treat malaria in the subject.

### BRIEF DESCRIPTION OF THE FIGURES

The patent or application file contains at least one figure executed in colour. Copies of this patent or patent application publication with colour figure(s) will be provided by the Office upon request and payment of the necessary fee.

Embodiments of the disclosure are described herein, by way of non-limiting example only, with reference to the following figures.

FIG. 1 provides a graphical representation of the workflow for the EasySep Human Glycophorin A Depletion kit. The Tetrameric Antibody Complex (TAC) is comprised of two mouse IgG1 antibodies directed against a surface marker of interest (e.g. glycophorin to label the RBC) and dextran (labelling the magnetic particle), respectively. This is held together by two unique linking antibodies that are anti-mouse (TAC linker).

FIG. 2 shows confocal images of (A) freshly prepared and (B) lyophilized F3 liposomes containing P. falciparum antigens. The arrows represent liposome aggregates. Scale is 20 μm.

FIG. 3 shows immunomagnetic depletion of red cell membranes from P. falciparum antigen extract. Red cell membrane separation from parasite antigen was undertaken using the EasySep™ Human Glycophorin A depletion kit (STEMCELL Technologies) according to the manufacturer's instructions. Representative overlay plots from two independent preparations showing differential staining patterns pre- and post-immunomagnetic depletion. (A) Anti-Glycophorin A FITC and (B) bisbenzimide hoechst staining for red cell membrane antigen and parasite DNA respectively.

FIG. 4 provides an evaluation of anti-human red blood cell antibodies in immunized mice by flow cytometry. Serum (1:10 dilution) was collected from mice (n=10) immunized with F3 liposomes containing 10⁷ P. falciparum antigens with or without red cell membranes or empty liposomes. The MFI of anti-mouse IgG AlexaFlour-488 labelling are shown for each group of mice. Data were analyzed using unpaired Mann-Whitney test expressed as mean±standard error of the mean (SEM). **P<0.002, ***P<0.001.

FIG. 5 shows total IgG responses in BALB/c mice immunised with F3 liposomes containing P. falciparum antigens. (A) P. falciparum 7G8-specific total IgG antibody responses in preabsorbed sera (dilution 1:6400). (B) Py17X-specific total IgG antibody responses (serum dilution 1:100) in mice immunised with freshly prepared F3 liposomes containing P. falciparum antigens with and without red cell membranes 7 days after the third immunisation. The antibody response presented as optical density (OD) reading at 450 nm wavelength was expressed as mean±standard error of the mean (SEM) (n=10 mice per group). Data were analysed using unpaired Mann-Whitney test to compare antibody responses in immunised mice to controls that received empty liposomes. Additionally, comparisons were also made preabsorbed and unabsorbed sera (A). *P<0.033, *** P<0.001.

FIG. 6 shows the gating strategy to identify antigen-experienced T cells following immunization. Activated CD4⁺ T cells were characterised as CD3⁺CD49d^{hi}CD11a^{hi} while CD8⁺ were CD3⁺CD8^{lo}CD11a^{hi}.

FIG. 7 shows peripheral blood T cell activation in BALB/c mice immunized with F3 liposomes containing P. falciparum antigens. Activation of (A) CD4⁺ or (B) CD8⁺ T cells in BALB/c mice immunized with F3 liposomes containing P. falciparum antigens with or without red cell membranes, (C) CD4⁺, (D) CD8⁺ T cell activation in BALB/c mice immunized with fresh or lyophilized F3 liposomes containing P. falciparum antigens 7 days after the third immunization. Data is expressed as mean±standard error of the mean (SEM) (n=10 mice per group). Data were analysed using unpaired Mann-Whitney test to compare T cell activation amongst immunized mice to controls that received empty liposomes. *P<0.033, *** P<0.001.

FIG. 8 shows splenocyte proliferative responses. (A) BALB/c mice immunized with F3 liposomes containing P. falciparum 7G8 antigens with or without red cell membranes. (B) BALB/c mice immunized with fresh or lyophilized liposomes containing P. falciparum 7G8 antigens. Spleen cells were incubated with Py17X parasitized red blood cells (pRBCs), P. falciparum 7G8 or UGMCB-009 lysate, P. knowlesi lysate, mouse normal red blood cells (nRBCs), human nRBCs, culture media or Concanavalin A (ConA). After 54 h, the cells were pulsed with tritiated thymidine and incubated for an additional 18 h. The mean proliferative response measured as counts per minute (CPM) was expressed as ±standard error of the mean (SEM) (n=3 mice per group). Data were analysed using unpaired t test to compare Py17X−/P. falciparum 7G8-/UGMCB-009−/P. knowlesi-specific proliferative responses to mouse nRBCs/human nRBC responses for each liposome formulation. *P<0.033, **P<0.002, ***P<0.001.

FIG. 9 provides graphical representations of monitoring of parasitaemia and disease severity of immunised BALB/c mice following challenge of immunised BALB/c mice with Py17X soluble antigen extracts delivered in liposome. (A) Parasitaemia and (B) Haemoglobin (g/L) up to 30 days post challenge of immunized BALB/c mice and (C) clinical scores up to 26 days post challenge of immunized BALB/c mice. Data were expressed as mean±SEM (n=7 mice per group). Indicates the number of mice that were euthanised. Grey lines: 10⁷ Py17X whole parasites+F3/PHAD liposomes; Red lines: 50 μg/dose Py17X soluble extract+F3/PHAD liposomes; Green lines: 100 μg/dose Py17X soluble extract+F3/PHAD liposomes; Blue lines: 200 μg/dose Py17X soluble extract+F3/PHAD liposomes; Pink lines: Empty liposomes.

FIG. 10 provides graphical representations of splenocyte proliferative responses in BALB/c mice immunized with liposomes containing red-cell depleted soluble Py17X antigens. Spleen cells were incubated with Concanavalin A (ConA); culture media; soluble Py17X extract derived from parasitized red blood cells (Py pRBCs); and mouse normal red blood cells (nRBCs). Experiments were performed with (A) empty liposomes; and liposomes containing red cell membrane-depleted Py17X antigens at (B) 50 μg/dose; (C) 100 μg/dose; (D) 200 μg/dose; and (E) 10⁷ μg/dose. After 54 hrs, the cells were pulsed with tritiated thymidine and incubated for an additional 18 hrs. The proliferative response measured as corrected counts per minute (CCPM) and expressed as mean±SEM (n=3 mice per group). Data were analysed using unpaired t test to compare proliferative responses following Py17X pRBC/P. falciparum 7G8 pRBC stimulation to mouse nRBC/human nRBC responses for each liposome formulation. *P<0.033, **P<0.01, ***P<0.002, ****P<0.001.

FIG. 11 provides a graphical representation of total IgG responses in mice immunised with mannosylated liposomes containing Py17X antigens without red cell components. (A) Py17X- and (B) P. falciparum 7G8-specific total IgG antibody responses (serum dilution 1:100) in BALB/c mice 7 days after the third immunisation. The antibody response presented as OD measured at 450 nm wavelength was expressed as mean±SEM. Data were analysed using unpaired Mann-Whitney U test to compare antibody responses in immunised mice to controls that received empty liposomes. *** P<0.001, ns: non-significant HIS: Py17X Hyperimmune serum used as positive control.

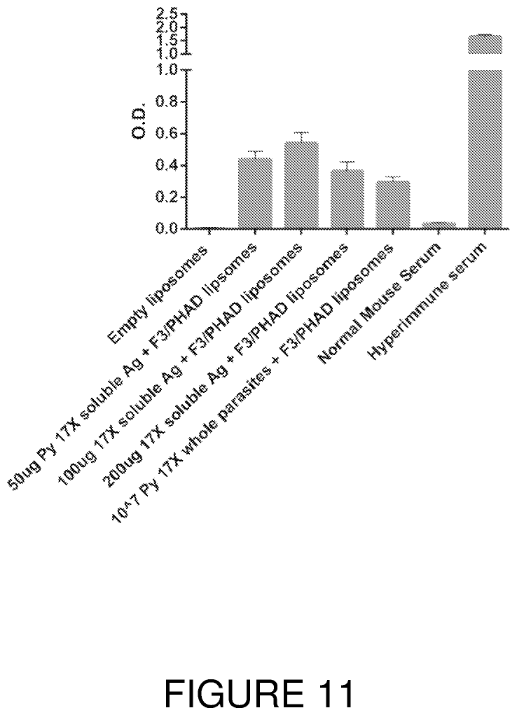

FIG. 12 provides graphical representations of monitoring of parasitaemia and disease severity of BALB/c mice immunised with 10⁷ P. falciparum soluble extract (RBC component depleted) in F3/PHAD liposomes, following challenge of immunised BALB/c mice with 10⁵ Py17X pRBC. (A) Parasitaemia and (B) Haemoglobin (g/L) up to 30 days post challenge of immunized BALB/c mice and (C) clinical scores up to 26 days post challenge of immunized BALB/c mice. Data were expressed as mean±SEM Indicates the number of mice that were euthanised.

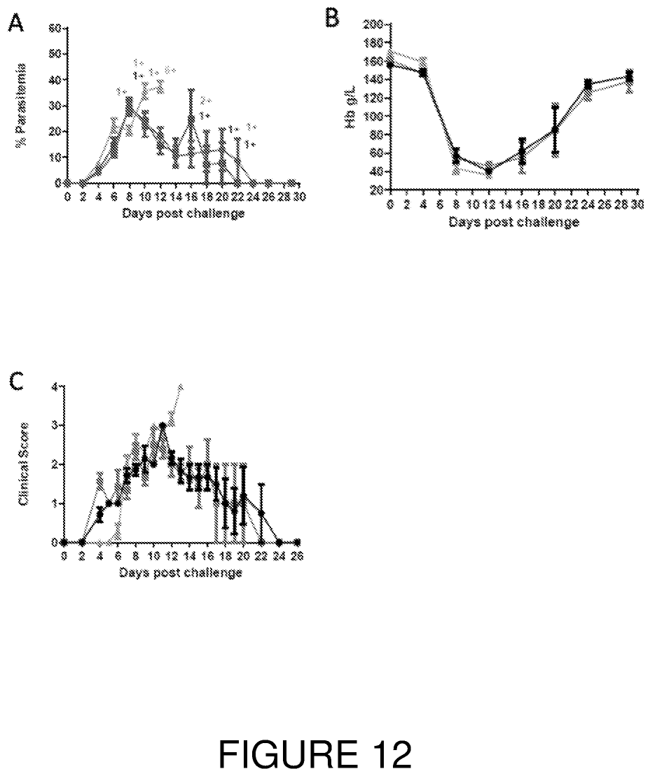

FIG. 13 provides graphical representations of splenocyte proliferative responses in BALB/c mice immunized with 10⁷ P. falciparum (A) soluble parasite extract (RBC component depleted) in F3/PHAD liposomes, (B) whole parasite lysate (RBC depleted) in F3/PHAD liposomes; or (C) empty liposomes. Spleen cells were incubated with 10⁷ P. falciparum whole parasites (RBC component depleted) in F3/PHAD liposomes with Concanavalin A (ConA); culture media; Py17X soluble extract derived from parasitized red blood cells (Py pRBCs); and mouse normal red blood cells (nRBCs) P. falciparum soluble extract derived from parasitized red blood cells (Pf pRBCs); and human nRBCs. Experiments were performed with (A) empty liposomes; and liposomes containing red cell membrane-depleted Py17X antigens at (B) 50 μg/dose; (C) 100 μg/dose; (D) 200 μg/dose; and (E) 10⁷ μg/dose. After 54 hrs, the cells were pulsed with tritiated thymidine and incubated for an additional 18 hrs. The proliferative response measured as corrected counts per minute (CCPM) and expressed as mean±SEM. Data were analysed using unpaired t test to compare proliferative responses following Py17X pRBC/P. falciparum 7G8 pRBC stimulation to mouse nRBC/human nRBC responses for each liposome formulation. *P<0.033, **P<0.01, ***P<0.002, ****P<0.001.

FIG. 14 provides a graphical representation of depletion of red cell membranes from P. falciparum antigen through processing which involved chemical lysis, freeze thawing and centrifugation with or without filtration. Representative overlay plots show two preparations of 10⁷ P. falciparum antigen that were produced using saponin lysis, freeze thawing and centrifugation (one with filtration and one without) to deplete red blood cell membranes compared with unprocessed 10⁷ P. falciparum (A) Anti-Glycophorin A FITC and (B) bisbenzimide hoechst staining for red cell membrane antigen and parasite DNA respectively.

FIG. 15 provides an evaluation of anti-human red blood cell antibodies in immunized mice by flow cytometry. Serum (1:10 dilution) was collected from mice (n=5/group) immunized with F3+ PHAD liposomes containing 10⁷ P. falciparum antigen with or without processing to deplete red cell membranes or empty liposomes. The MFI of anti-mouse IgG labelling are shown for each group of mice. Each dot represents an individual mouse and the line and error bars for each group represent the mean±SEM.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” or “approximate” and their grammatically equivalent expressions is meant a quantity, level, value, number, frequency, percentage, dimension, size, amount, abundance, concentration, weight or length that varies by as much 15, 14, 13, 12, 11, 10, 9, 8, 7, 6, 5, 4, 3, 2 or 1% to a reference quantity, level, value, number, frequency, percentage, dimension, size, amount, abundance, concentration, weight or length.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and preferably within less than about one to about four hours. When administered contemporaneously, the agents are suitably administered at the same site on the subject. The term “same site” includes the exact location, but can be within about 0.5 to about 15 centimetres, preferably from within about 0.5 to about 5 centimetres. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

By “antigen” is meant all, or part of, a molecule (e.g., a protein, peptide, or other molecule or macromolecule) capable of being bound by an antibody or a T-cell receptor (TCR) if presented by MHC molecules An antigen may be additionally capable of being recognized by the immune system and/or being capable of stimulating or inducing a humoral immune response and/or cellular immune response leading to the activation of B- and/or T-lymphocytes. An antigen may have one or more epitopes (B- and T-epitopes). Antigens as used herein may also be mixtures of several individual antigens.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “effective amount”, in the context of modulating an immune response or treating or preventing a disease or condition, is meant the administration of that amount of composition to an individual in need thereof, either in a single dose or as part of a series, that is effective for that modulation, treatment or prevention. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As generally used herein the terms “immunize”, “vaccinate” and “vaccine” refer to methods and/or formulations that elicit a protective immune response against a parasite, whereby subsequent infection by the parasite is at least partly prevented or minimized.

In the context of the present invention, the term “immunogenic” as used herein indicates the ability or potential to generate or elicit an immune response, such as to a pathogen or molecular components thereof, upon administration of the immunogenic agent to a mammal or other animal.

As used herein, the term “immunostimulatory composition” refers to a composition or formulation which contains the composition of the present invention and which is in a form that is capable of being administered to any vertebrate, preferably an animal, preferably a mammal, and more preferably a human. Optionally, the immunostimulatory composition includes or is prepared from dry powder in a conventional saline or buffered aqueous solution medium in which the composition of the present invention is suspended or dissolved. In this form, the composition of the present invention can be used conveniently to prevent, ameliorate, manage or otherwise treat a condition. Upon introduction into a host, the immunostimulatory composition is able to elicit an immune response, preferably a detectable immune response, including, but not limited to, the production of antibodies, cytokines and/or the activation of cytotoxic T-cells, antigen presenting cells, helper T-cells, dendritic cells and/or other cellular responses. The immunostimulatory composition of the present invention includes soluble parasite antigens. Immunostimulatory compositions of the present invention may include or be administered in or with an adjuvant.

By “isolated” is meant material that is substantially or essentially free from components that normally accompany it in its native state. Similarly, an “isolated” or “purified” parasite (e.g., malaria parasite) is substantially free of cellular material or other contaminating molecules from the cell type or tissue source from which the parasite was derived, or substantially free from chemical precursors or other chemicals when chemically synthesized. “Substantially free” means that a preparation of soluble parasite antigen is at least 10, 15, 20, 25, 30, 35, 40, 45, 50, 55, 60, 65, 70, 75, 80, 85, 90, 91, 92, 93, 94, 95, 96, 97, 98 or 99% pure. In a preferred embodiment, the preparation of soluble parasite antigen has less than about 30, 25, 20, 15, 10, 9, 8, 7, 6, 5, 4, 3, 2 or 1% (by dry weight), of insoluble parasite antigen, of non-parasite antigen (also referred to herein as a “contaminating molecules”), or of chemical precursors or non-parasite antigen chemicals. The soluble parasite antigen is also desirably substantially free of culture medium, i.e., culture medium represents less than about 20, 15, 10, 5, 4, 3, 2, 1% of the volume of the soluble parasite extract. The invention includes isolated or purified preparations of at least 0.01, 0.1, 1.0, and 10 milligrams in dry weight.

By “modulating” is meant increasing or decreasing, either directly or indirectly, the immune response of an individual. In certain embodiments, “modulation” or “modulating” means that a desired/selected response (e.g., a tolerogenic or anergic response) is more efficient (e.g., at least 10%, 20%, 30%, 40%, 50%, 60% or more), more rapid (e.g., at least 10%, 20%, 30%, 40%, 50%, 60% or more), greater in magnitude (e.g., at least 10%, 20%, 30%, 40%, 50%, 60% or more), and/or more easily induced (e.g., at least 10%, 20%, 30%, 40%, 50%, 60% or more) than in the absence of the immunomodulatory composition.

By “obtained from” is meant that a sample such as, for example, a polypeptide extract is isolated from, or derived from, a particular source.

As used herein, the term “parasite extract” means one or more of malaria parasites that have been isolated, enriched or otherwise fractionated either from an external environment or for a particular fraction of the parasite (e.g., the soluble fraction of the parasite).

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of stimulating or inducing an antigen-specific Th1 response, or in need of treatment or prophylaxis of an infectious disease or condition, including parasitic diseases (e.g., malaria) which are often associated with the presence or aberrant expression of an antigen of interest. However, it will be understood that the aforementioned terms do not imply that symptoms are present.

By “pharmaceutically acceptable carrier” is meant a solid or liquid filler, diluent or encapsulating substance that can be safely used in topical or systemic administration to an animal, preferably a mammal, including humans.

By “Plasmodium parasite” or “Plasmodium organism” is meant any member of the protozoan genus Plasmodium, including any of the species that cause human malaria including Plasmodium falciparum, Plasmodium vivax, Plasmodium malariae, and Plasmodium ovale, Plasmodium knowlesi, Plasmodium berghi, Plasmodium yoelii, Plasmodium chabaudi, and Plasmodium vinckei.

“Polypeptide”, “peptide”, “protein” and “proteinaceous molecule” are used interchangeably herein to refer to molecules comprising or consisting of a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

As used herein, “preventing”, “prevent” or “prevention” refers to a course of action initiated prior to infection by, or exposure to, a malaria parasite and/or before the onset of a symptom or pathological sign of malaria or an associated disease, disorder or condition, so as to prevent infection and/or reduce the symptom or pathological sign. It is to be understood that such preventing need not be absolute to be beneficial to a subject. A “prophylactic” treatment is a treatment administered to a subject who does not exhibit signs of the disease, disorder or condition, or exhibits only early signs for the purpose of decreasing the risk of developing a symptom or pathological sign of the disease, disorder or condition.

By “substantially free” is meant that the described feature is less than 20%, less than 15%, less than 10%, less than 5%, 4%, 3%, 2%, 1%, 0.5%, 0.2% or 0.1% of that which was present in a native environment, on a w/w basis. For example, the proportion of RBC components present after depletion would be less that 15%, less than 10%, less than 5%, 4%, 3%, 2%, 1%, 0.5%, 0.2% or 0.1% of that which was present prior to the depletion of RBC components.

The term “Th1” refers to a subclass of T helper cells that produce inter alia IL-1, IL-2, IL-8, IL-12, IL-18, interferon-gamma (IFN-γ), tumor necrosis factor-alpha (TNF-α), and which elicit inflammatory reactions associated with a cellular, i.e., non-immunoglobulin, response to a challenge. Thus, a Th1 cytokine response or T1 cytokine response encompasses an immune response whose most prominent feature comprises abundant CD4⁺ helper T-cell activation that is associated with increased levels of T1 cytokines (e.g., IL-1, IL-2, IL-8, IL-12, IL-18, IFN-γ, TNF-α, etc.) relative to these cytokine amounts in the absence of activation. A T1 cytokine response can also refer to the production of T1 cytokines from other white blood cells and nonwhite blood cells. A Th1 cytokine response can include abundant CD8 cytotoxic T lymphocyte activity including T1 cytokine production, referred to as Tc1. A Th1 response is typically promoted by CD4 “Th1” T-helper cells however a Th1 response can include CD8 Tc1 T cytotoxic cells.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effects attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

### 2. Abbreviations

The following abbreviations are used throughout the application:
aa=amino acid(s)
kDa=kilodalton(s)
d=day
h=hour
s=seconds
APC=antigen-presenting cell
RBC=red blood cell

### 3. Parasite Antigen Preparations

The present invention is predicated at least in part on the determination that soluble parasite extracts when administered to a subject are surprisingly effective at stimulating an immune response against a parasite. The present inventors thus consider that these parasite extracts described herein will be useful in formulating immunostimulating compositions to generate immune responses (e.g., cellular immune responses) in animals for treating or preventing parasite-associated disease, including malaria.

Accordingly, the present invention provides soluble parasite extracts in methods and compositions for treating or preventing parasitic disease in a subject. When included in compositions, the soluble parasite extracts are suitably combined with is pharmaceutically acceptable carriers or diluents. The parasite extracts of the present invention can be administered by any suitable route including for example, by injection, to treat or prevent a parasitic disease in a subject.

In some embodiments, the parasite extracts are obtained from a parasite of the apicomplexan phyla, non-limiting examples of which include the parasites of the genus Plasmodium (e.g., Plasmodium falciparum, P. malariae, P. ovale, P. vivax, and P. knowlesi); parasites of the genus Babesia (e.g., Babesia bigemina, B. bovis, B. caballi, B. canis, B. divergens, B. microti, and B. motasi); parasites of the genus Cryptosporidiosis (e.g., Cryptosporidium parvum); Cyclosporiasis (e.g., Cyclospora cayetanensis); Cystoisosporiasis e.g., Cystoisospora belli); and Toxoplasmosis (e.g., Toxoplasma gondii).

In some preferred embodiments, the parasite extract comprises Plasmodium falciparum, Plasmodium vivax, Plasmodium malariae, and Plasmodium ovale, Plasmodium knowlesi, Plasmodium berghi, Plasmodium yoelii, Plasmodium chabaudi, and Plasmodium vinckei. In some preferred embodiments, the parasite extract is derived from a Plasmodium falciparum parasite.

In some embodiments, the parasite extract comprises a soluble fraction from two or more species of parasite. In some embodiments, the parasite extract comprises a soluble fraction from three or more species of parasite. In some embodiments, the parasite extract comprises a soluble fraction from four or more species of parasite.

In some of the same embodiments and some other embodiments, the parasite extract is made using blood-stage parasites. (e.g., merozoites, schizonts, rings or trophozoites, although without limitation thereto). In some preferred embodiments, the parasites are early “ring” stage parasites.

In some of the same embodiments and in some other embodiments, the malaria parasites are inactivated while in the red blood cells (RBC) and are then subsequently extracted. The inactivation may be by way of attenuation of the vaccine (e.g., chemical attenuation). Methods of attenuating parasites are known in the art and described, for example, in Raja et al., 2017.

In some embodiments the parasite extract is at least partially purified from RBC components. The parasite extract is typically substantially free of any RBC components (including cell membranes and fragments thereof, membrane proteins, and soluble proteins). As described above, the inclusion of RBC components allows a host that is administered the parasite extract to generate auto-antibodies to, and subsequently elicit an immune response against, the RBC components present in the extract. Accordingly, the removal of any RBC components ensures an effective immune response to parasite antigens, and prevents stimulating an autoreactive immune response by the host.

In some embodiments the parasite extract is at least partially purified from RBC membranes. In some particularly preferred embodiments the parasite extract is substantially free of RBC membranes. In some of the same embodiments and some other embodiments, the parasite extract is substantially free of soluble RBC proteins. In some preferred embodiments, the parasite extract is at least 98%, 99%, 99.5%, 99.6%, 99.7%, 99.8%, or 99.9% free of RBC components.

In some preferred embodiments, the parasite extract comprises, consists, or consists essentially of the soluble parasite components (i.e., being substantially free from insoluble parasite components). In some of the same embodiments and some other embodiments, the parasite extract is substantially free from insoluble parasite membranes.

Alternatively, the parasite extract may be isolated from all RBC components, but comprise all the parasite fractions (i.e., soluble fraction and insoluble fraction). In embodiments of this type, the parasite extract generally comprises a whole parasite lysate.

### 4. Compositions for Stimulating an Immune Response

The present inventors have determined that soluble parasite extract has the ability to stimulate an immune response to a parasite antigen in vivo. These soluble parasite extract can be administered in soluble form, in particulate form, and/or in the form of antigen-presenting cells that have been contacted ex vivo with the soluble parasite antigen preparations.

In some embodiments, the parasite antigen preparations are targeted to a particular region or cell type. For example, in some embodiments, the parasite extract is targeted to an immune cell (e.g., an APC).

### 4.1 Particle Embodiments

In some embodiments, the soluble parasite extracts as discussed above and/or elsewhere herein, are provided in particulate form. A variety of particles may be used in the invention, including but not limited to liposomes, micelles, lipidic particles, ceramic/inorganic particles and polymeric particles. In some preferred embodiments, the parasite extract is formulated into a lipid vesicle, and preferable a liposome.

In illustrative examples, the particles have a dimension of less than about 100 μm, more suitably in the range of less than or equal to about 500 nm, although the particles may be as large as about 10 μm, and as small as a few nm. Liposomes consist basically of a phospholipid bilayer forming a shell around an aqueous core. Advantages include the lipophilicity of the outer layers which “mimic” the outer membrane layers of cells and that they are taken up relatively easily by a variety of cells. Polymeric vehicles typically consist of micro/nanospheres and micro/nanocapsules formed of biocompatible polymers, which are either biodegradable (for example, polylactic acid) or non-biodegradable (for example, ethylenevinyl acetate). Some of the advantages of the polymeric devices are ease of manufacture and high loading capacity, range of size from nanometre to micron, as well as controlled release and degradation profiles.

In some embodiments, the particles comprising an antigen-binding molecule on their surface, which is immuno-interactive with a marker that is expressed at higher levels on antigen-presenting cells (e.g., dendritic cells) than on non-antigen-presenting cells. Illustrative markers of this type include MGL, CDL-1, DEC-205, macrophage mannose R, DC-SIGN or other dendritic cell or myeloid specific (lectin) receptors, as for example disclosed by Hawiger et al., (2001, J Exp Med 194, 769), Kato et al., (2003, J Biol Chem 278, 34035), Benito et al., (2004, J Am Chem Soc 126, 10355), Schjetne et al., (2002, Int Immunol 14, 1423), van Vliet et al., (2006, Nat Immunol 7(11): 1200-8), van Vliet et al., (2006, Immunobiology 211, 577-585).

Antigen-presenting cells (APCs) include both professional and facultative types of APCs. Professional APCs include, but are not limited to, macrophages, monocytes, B lymphocytes, cells of myeloid lineage, including monocytic-granulocytic-DC precursors, marginal zone Kupffer cells, microglia, T cells, Langerhans cells and dendritic cells including interdigitating dendritic cell and follicular dendritic cells. In some embodiments, the APC is selected from the group comprising monocytes, macrophages, B-lymphocytes, cells of myeloid lineage, dendritic cells, and Langerhans cells.

The particles can be prepared from a parasite extract and optionally, an adjuvant, surfactant, excipient, or polymeric material. In some embodiments, the particles are biodegradable and biocompatible, and optionally are capable of biodegrading at a controlled rate for delivery of a therapeutic or diagnostic agent. The particles can be made of a variety of materials. Both inorganic and organic materials can be used. Polymeric and non-polymeric materials, such as fatty acids, may be used. Other suitable materials include, but are not limited to gelatin, polyethylene glycol, trehalulose, dextran, and chitosan. Particles with degradation and release times ranging from seconds to months can be designed and fabricated, based on factors such as the particle material.

Lipid Vesicles

Suitably, the RBC-depleted, parasite extract is formulated into a lipid vesicle. As broadly used herein, the lipid vesicle may be a liposome, minicell, multilamellar vesicle, micelle, vacuole or other vesicular structure comprising a lipid bilayer. Parasite extracts may be located in the intravesicular space or may be displayed on the liposome surface.

The lipid vesicle suitably comprises any lipid or mixture of lipids capable of forming a lipid bilayer structure. These include a phospholipids, sterols inclusive of cholesterol, cholesterol-esters and phytosterols, fatty acids and/or triglycerides. Non-limiting examples of phospholipids include phosphatidylcholine (PC) (lecithin), phosphatidic acid, phosphatidylethanolamine (PE) (cephalin), phosphatidylglycerol (PG), phosphatidylserine (PS), phosphatidylinositol (PI) and sphingomyelin (SM) or natural or synthetic derivatives thereof. Natural derivatives include egg PC, egg PG, soy bean PC, hydrogenated soy bean PC, soy bean PG, brain PS, sphingolipids, brain SM, galactocerebroside, gangliosides, cerebrosides, cephalin, cardiolipin, and dicetylphosphate. Synthetic derivatives include 1,2-dipalmitoyl-sn-glycero-3-phosphocholine (DPPC), dimethyldioctadecylammonium bromide (DDAB), didecanoylphosphatidylcholine (DDPC), dierucoylphosphatidylcholine (DEPC), dimyristoylphosphatidylcholine (DMPC), distearoylphosphatidylcholine (DSPC), dilaurylphosphatidylcholine (DLPC), palmitoyloleoylphosphatidylcholine (POPC), palmitoylmyristoylphosphatidylcholine (PMPC), palmitoylstearoylphosphatidylcholine (PSPC), dioleoylphosphatidylcholine (DOPC), dioleoylphosphatidylethanolamine (DOPE), dilauroylphosphatidylglycerol (DLPG), distearoylphosphatidylglycerol (DSPG), dimyristoylphosphatidylglycerol (DMPG), dipalmitoylphosphatidylglycerol (DPPG), distearoylphosphatidylglycerol (DSPG), dioleoylphosphatidylglycerol (DOPG), palmitoyloleoylphosphatidylglycerol (POPG), dimyristoylphosphatidic acid (DMPA), dipalmitoylphosphatidic acid (DPPA), distearoylphosphatidic acid (DSPA), dimyristoylphosphatidylethanolamine (DMPE), dipalmitoylphosphatidylethanolamine (DPPE), dimyristoylphosphatidylserine (DMPS), dipalmitoylphosphatidylserine (DPPS), distearoylphosphatidylethanolamine (DSPE), dioleoylphosphatidylethanolamine (DOPE) dioleoylphosphatidylserine (DOPS), dipalmitoylsphingomyelin (DPSM) and distearoylsphingomyelin (DSSM). The phospholipid can also be a derivative or analogue of any of the above phospholipids.

In a preferred embodiment, the lipid vesicle is a liposome.

The particle size of liposomes has also been shown to impact adjuvanticity and direct the development of the resulting cell-mediated immune response. Studies have shown that the immune response induced following administration of small sized liposome vesicles was skewed towards Th2 while larger vesicles induced a Th1 response characterised by augmented IFN-γ and IgG2a production (see, Mann et al., 2009). The differences in the profiles of the induced immune response of large verses small vesicles could be due to differences in antigen processing and trafficking to lymph nodes. Large sized vesicles were shown to be more efficiently phagocytosed and processed by macrophages compared to smaller vesicles (Brewer et al., 2004). Additionally, trafficking of liposome particles to lymph nodes has been shown to be size dependent with small sized vesicles freely draining to and specifically targeting lymph node-resident cells while large sized vesicles require dendritic cells for trafficking from the injection site to lymph nodes (see, Manolova et al., 2008). More recently, using cationic liposomes, large sized vesicles demonstrated enhanced splenocyte proliferative responses and reduced IL-10 production compared to small sized liposomes (see, Henriksen-Lacey et al., 2011). In some embodiments of the present invention, the volume-weighted diameter of freshly prepared liposomes is between about 10 μm and about 100 μm, about 20 μm and about 80 μm, and about 20 μm to about 50 μm. The volume weighted diameter of lyophilized liposomes generally between about 25 μm and about 150 μm, 40 μm and about 100 μm, or about 50 μm and 75 μm. In some of the same embodiments and some other embodiments, the size distribution of the liposomes is generally <10. Preferably, the span is <5.

In some preferred embodiments, the liposomes comprise, consist, or consist essentially of 1,2-dipalmitoyl-sn-glycero-3-phosphocholine (DPPC), dimethyldioctadecylammonium bromide (DDAB) and cholesterol, in the ratio of 5:2:1. In some alternative preferred embodiments the liposomes comprise, consist, or consist essentially of 1,2-dipalmitoyl-sn-glycero-3-phosphocholine (DPPC), dimethyldioctadecylammonium bromide (DDAB) and cholesterol, in the ratio of 7:2:1.

In some embodiments the liposome is combined with an immunostimulatory adjuvant to create liposome adjuvant systems. Suitable examples of such adjuvant systems include those selected from the group comprising CAF01 (liposomes plus DDA plus TDB), cationic liposomal vaccine adjuvant, Stealth liposomes, JVRS-100 (cationic liposomal DNA complex), cytokine-containing liposomes, immunoliposomes containing antibodies to costimulatory molecules, DRVs (immunoliposomes prepared from dehydration-rehydration vesicles), MTP-PE liposomes, Sendai proteoliposomes, Sendai containing lipid matrices, Walter Reed liposomes (liposomes containing lipid A adsorbed to aluminium hydroxide), AS01 (MPL plus liposome plus QS-21), and AS15 (MPL plus CpG plus QS-21 plus liposome).

In some particularly preferred embodiments, the immunostimulatory compositions of the invention comprise the liposome adjuvant system, CAF01. CAF01 has advantageous immunological properties and high stability profile, which makes make it particularly suitable for vaccine formation for the developing world, which in addition to vaccine efficacy are important prerequisites.

In some embodiments, the particle may comprise a virosome compound (unilamellar liposomal vehicles incorporating virus derived proteins, such as influenza haemagglutinin), e.g. IRIVs (immunopotentiating reconstituted influenza virosomes), or liposomes of lipids plus hemagglutinin. In some embodiments the particle may be selected from a virus-like particle (VLP), e.g., Ty particles (Ty-VLPs), albumin-heparin microparticles, or CRL1005 (block copolymer P1205), peptomere nanoparticle, CAP™ (calcium phosphate nanoparticles), microspheres, PODDS® (proteinoid microspheres), and nanospheres. Mo

A particle carrying a payload of parasite extract(s) can be made using the procedure as described in: Pautot et al., (2003, Proc. Natl. Acad. Sci. USA 100(19): 10718-21). Using the Pautot et al., technique, streptavidin-coated lipids (DPPC, DSPC, and similar lipids) can be used to manufacture liposomes. The drug encapsulation technique described by Needham et al., (2001, Advanced Drug Delivery Reviews 53(3): 285-305) can be used to load these vesicles with one or more antigens.

The liposomes can be prepared by exposing chloroformic solution of various lipid mixtures to high vacuum and subsequently hydrating the resulting lipid films (DSPC/CHOL) with pH 4 buffers, and extruding them through polycarbonated filters, after a freezing and thawing procedure. It is possible to use DPPC supplemented with DSPC or cholesterol to increase encapsulation efficiency or increase stability, etc. A transmembrane pH gradient is created by adjusting the pH of the extravesicular medium to 7.5 by addition of an alkalinization agent. A parasite antigen preparation and optionally any adjuvants or other immunostimulatory molecules may be subsequently entrapped by addition of a solution of the parasite extract in small aliquots to the vesicle, solution, at an elevated temperature, to allow accumulation of the antigenic preparations inside the liposomes.

Other lipid-based particles suitable for the delivery of the bioactive agents of the present invention such as niosomes are described by Copeland et al., (2005, Immunol. Cell Biol. 83: 95-105).

In some embodiments, the lipid vesicles (e.g., liposomes) are lyophilized in order to assist with storage and delivery. Methods of lyophilising liposomes are known in the art, as disclosed by Zaman et al., 2016 (the contents of which is included herein by reference). In some embodiments, trehalose is used as a lyoprotectant. In embodiments, of this type, the lyophilised liposomes are resuspended in any suitable buffer prior to administration. Typically, the lyophilised liposomes are resuspended in PBD prior to administration.

Polymeric Particles

Polymeric particles may be formed from any biocompatible and desirably biodegradable polymer, copolymer or blend. The polymers may be tailored to optimize different characteristics of the particle including: (i) interactions between the parasite extract to be delivered and the polymer to provide stabilization of the parasite extract and retention of the immunogenicity upon delivery; (ii) rate of polymer degradation and, thereby, rate of agent release profiles; (iii) surface characteristics and targeting capabilities via chemical modification; and (iv) particle porosity.

Surface eroding polymers, such as polyanhydrides may be used to form the particles. For example, polyanhydrides such as poly[(p-carboxyphenoxy)-hexane anhydride] (PCPH) may be used. Biodegradable polyanhydrides are described in U.S. Pat. No. 4,857,311.

In other embodiments, bulk eroding polymers such as those based on polyesters including poly(hydroxy acids) or polyesters can be used. For example, polyglycolic acid (PGA), polylactic acid (PLA), or copolymers thereof may be used to form the particles. The polyester may also have a charged or functionalizable group, such as an amino acid. In illustrative examples, particles with controlled release properties can be formed of poly(D, L-lactic acid) and/or poly(D, L-lactic-co-glycolic acid) (“PGLA”) which incorporate a surfactant such as DPPC.

Other polymers include poly(alkycyanacrylates), polyamides, polycarbonates, polyalkylenes such as polyethylene, polypropylene, poly(ethylene glycol), poly(ethylene oxide), poly(ethylene terephthalate), poly vinyl compounds such as polyvinyl alcohols, polyvinyl esters, polymers of acrylic and methacrylic acids, celluloses and other polysaccharides, and peptides or proteins, or copolymers or blends thereof. Polymers may be selected with or modified to have the appropriate stability and degradation rates in vivo for different controlled drug delivery applications.

In some embodiments, particles are formed from functionalized polyester-graft copolymers, as described in Hrkach et al., (1995, Macromolecules 28: 4736-4739; and “Poly(L-Lactic acid-co-amino acid) Graft Copolymers: A Class of Functional Biodegradable Biomaterials” in Hydrogels and Biodegradable Polymers for Bioapplications, ACS Symposium Series No. 627, Raphael M. Ottenbrite et al, Eds., American Chemical Society, Chapter 8, pp. 93-101, 1996.)

Materials other than biodegradable polymers may be used to form the particles. Suitable materials include various non-biodegradable polymers and various excipients. The particles also may be formed of the bioactive agent(s) and surfactant alone.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art, provided that the conditions are optimized for forming particles with the desired diameter.

Methods developed for making microspheres for delivery of encapsulated agents are described in the literature, for example, as described in Doubrow, M., Ed., “Microcapsules and Nanoparticles in Medicine and Pharmacy,” CRC Press, Boca Raton, 1992. Methods also are described in Mathiowitz and Langer (1987, J. Controlled Release 5, 13-22); Mathiowitz et al., (1987, Reactive Polymers 6, 275-283); and Mathiowitz et al., (1988, J. Appl. Polymer Sci. 35, 755-774) as well as in U.S. Pat. Nos. 5,213,812, 5,417,986, 5,360,610, and 5,384,133. The selection of the method depends on the polymer selection, the size, external morphology, and crystallinity that is desired, as described, for example, by Mathiowitz et al., (1990, Scanning Microscopy 4: 329-340; 1992, J. Appl. Polymer Sci. 45, 125-134); and Benita et al., (1984, J. Pharm. Sci. 73, 1721-1724).

In solvent evaporation, described for example, in Mathiowitz et al., (1990), Benita; and U.S. Pat. No. 4,272,398, the polymer is dissolved in a volatile organic solvent, such as methylene chloride. Several different polymer concentrations can be used, for example, between about 0.05 and about 2.0 g/mL. The bioactive agent(s), either in soluble form or dispersed as fine particles, is (are) added to the polymer solution, and the mixture is suspended in an aqueous phase that contains a surface-active agent such as poly (vinyl alcohol). The aqueous phase may be, for example, a concentration of 1% poly(vinyl alcohol) w/v in distilled water. The resulting emulsion is stirred until most of the organic solvent evaporates, leaving solid microspheres, which may be washed with water and dried overnight in a lyophilizer. Microspheres with different sizes (between 1 and 1000 μm) and morphologies can be obtained by this method.

Solvent removal was primarily designed for use with less stable polymers, such as the polyanhydrides. In this method, the agent is dispersed or dissolved in a solution of a selected polymer in a volatile organic solvent like methylene chloride. The mixture is then suspended in oil, such as silicon oil, by stirring, to form an emulsion. Within 24 hours, the solvent diffuses into the oil phase and the emulsion droplets harden into solid polymer microspheres. Unlike the hot-melt microencapsulation method described for example in Mathiowitz et al., (1987, Reactive Polymers 6:275), this method can be used to make microspheres from polymers with high melting points and a wide range of molecular weights. Microspheres having a diameter for example between one and 300 microns can be obtained with this procedure.

With some polymeric systems, polymeric particles prepared using a single or double emulsion technique, vary in size depending on the size of the droplets. If droplets in water-in-oil emulsions are not of a suitably small size to form particles with the desired size range, smaller droplets can be prepared, for example, by sonication or homogenation of the emulsion, or by the addition of surfactants.

If the particles prepared by any of the above methods have a size range outside of the desired range, particles can be sized, for example, using a sieve, and further separated according to density using techniques known to those of skill in the art.

The polymeric particles can be prepared by spray drying. Methods of spray drying, such as that disclosed in International PCT Publication No. WO 96/09814, disclose the preparation of smooth, spherical microparticles of a water-soluble material with at least 90% of the particles possessing a mean size between about 1 and about 10 μm.

Ceramic Particles

Ceramic particles may also be used to deliver the bioactive agents of the invention. These particles are typically prepared using processes similar to the well known sol-gel process and usually require simple and room temperature conditions as described for example in Brinker et al., (“Sol-Gel Science: The Physics and Chemistry of Sol-Gel Processing;” Academic Press: San Diego, 1990, 60), and Avnir et al., (1994, Chem. Mater. 6, 1605), Ceramic particles can be prepared with desired size, shape and porosity, and are extremely stable. These particles also effectively protect doped molecules (polypeptides, drugs etc.) against denaturation induced by extreme pH and temperature (Jain et al., 1998, J. Am. Chem. Soc. 120, 11092-11095). In addition, their surfaces can be easily functionalized with different groups (Lai et al., 2000, Chem. Mater. 12, 2632-2639; Badley et al., 1990, Langmuir 6, 792-801), and therefore they can be attached to a variety of monoclonal antibodies and other ligands in order to target them to desired sites in vivo.

Various ceramic particles have been described for delivery in vivo of active agent-containing payloads. For example, British Patent No. 1,590,574 discloses incorporation of biologically active components in a sol-gel matrix. International PCT Publication No. WO 97/45367 discloses controllably dissolvable silica xerogels prepared via a sol-gel process, into which a biologically active agent is incorporated by impregnation into pre-sintered particles (1 to 500 μm) or disks. International Patent Publication No. WO 2000/050349 discloses controllably biodegradable silica fibres prepared via a sol-gel process, into which a biologically active agent is incorporated during synthesis of the fibre. U.S. Patent Application Publication No. 2004/0180096 describes ceramic nanoparticles in which a bioactive substance is entrapped. The ceramic nanoparticles are made by formation of a micellar composition of the dye. The ceramic material is added to the micellar composition and the ceramic nanoparticles are precipitated by alkaline hydrolysis. U.S. Patent Application Publication No. 2005/0123611 discloses controlled release ceramic particles comprising an active material substantially homogeneously dispersed throughout the particles. These particles are prepared by mixing a surfactant with an apolar solvent to prepare a reverse micelle solution; (b) dissolving a gel precursor, a catalyst, a condensing agent and a soluble active material in a polar solvent to prepare a precursor solution; (c) combining the reverse micelle solution and the precursor solution to provide an emulsion and (d) condensing the precursor in the emulsion. U.S. Patent Application Publication No. 2006/0210634 discloses adsorbing bioactive substances onto ceramic particles comprising a metal oxide (e.g., titanium oxide, zirconium oxide, scandium oxide, cerium oxide and yttrium oxide) by evaporation. Kortesuo et al., (2000, Int J Pharm. 200(2): 223-229) disclose a spray drying method to produce spherical silica gel particles with a narrow particle size range for controlled delivery of drugs such as toremifene citrate and dexmedetomidine HCl. Wang et al., (2006, Int J Pharm. 3081(2): 160-167) describe the combination of adsorption by porous CaCO₃ microparticles and encapsulation by polyelectrolyte multilayer films for delivery of bioactive substances.

Ballistic Particles

The parasite extracts of the present invention may be attached to (e.g., by coating or conjugation) or otherwise associated with particles suitable for use in needleless or “ballistic” (biolistic) delivery. Illustrative particles for ballistic delivery are described, for example, in: International PCT Publication Nos. WO 02/101412; WO 02/100380; WO 02/43774; WO 02/19989; WO 01/93829; WO 01/83528; WO 00/63385; WO 00/26385; WO 00/19982; WO 99/01168; WO 98/10750; and WO 97/48485. It shall be understood, however, that such particles are not limited to their use with a ballistic delivery device and can otherwise be administered by any alternative technique (e.g., injection or microneedle delivery) through which particles are deliverable to immune cells.

The parasite extracts can be coated or chemically coupled to carrier particles (e.g., core carriers) using a variety of techniques known in the art. Carrier particles are selected from materials which have a suitable density in the range of particle sizes typically used for intracellular delivery. The optimum carrier particle size will, of course, depend on the diameter of the target cells. Illustrative particles have a size ranging from about 0.01 to about 250 μm, from about 10 to about 150 μm, and from about 20 to about 60 μm; and a particle density ranging from about 0.1 to about 25 g/cm³, and a bulk density of about 0.5 to about 3.0 g/cm³, or greater. Non-limiting particles of this type include metal particles such as, tungsten, gold, platinum and iridium carrier particles. Tungsten particles are readily available in average sizes of about 0.5 to about 2.0 μm in diameter. Gold particles or microcrystalline gold (e.g., gold powder A1570, available from Engelhard Corp., East Newark, N.J.) may also be used. Gold particles provide uniformity in size (available from Alpha Chemicals in particle sizes of about 1 to about 3 μm, or available from Degussa, South Plainfield, N.J. in a range of particle sizes including 0.95 μm) and low toxicity. Microcrystalline gold provides a diverse particle size distribution, typically in the range of about 0.1 to about 5 μm. The irregular surface area of microcrystalline gold provides for highly efficient coating with the active agents of the present invention.

Many methods are known and have been described for adsorbing, coupling or otherwise attaching bioactive molecules (e.g., soluble molecules such as proteins) onto particles such as gold or tungsten particles. In illustrative examples, such methods combine a predetermined amount of gold or tungsten with the parasite extracts, CaCl₂) and spermidine. In other examples, ethanol is used to precipitate the parasite extracts onto gold or tungsten particles (see, for example, Jumar et al., 2004, Phys Med. Biol. 49:3603-3612). The resulting solution is suitably vortexed continually during the coating procedure to ensure uniformity of the reaction mixture. After attachment of the antigenic preparation, the particles can be transferred for example to suitable membranes and allowed to dry prior to use, coated onto surfaces of a sample module or cassette, or loaded into a delivery cassette for use in particular particle-mediated delivery instruments.

The formulated compositions may suitably be prepared as particles using standard techniques, such as by simple evaporation (air drying), vacuum drying, spray drying, freeze drying (lyophilization), spray-freeze drying, spray coating, precipitation, supercritical fluid particle formation, and the like. If desired, the resultant particles can be dandified using the techniques described in International PCT Publication No. WO 97/48485.

4.2 Surfactants

Surfactants which can be incorporated into particles include phosphoglycerides. Exemplary phosphoglycerides include phosphatidylcholines, such as the naturally occurring surfactant, L-a-phosphatidylcholine dipalmitoyl (“DPPC”). The surfactants advantageously improve surface properties by, for example, reducing particle-particle interactions, and can render the surface of the particles less adhesive. The use of surfactants endogenous to the lung may avoid the need for the use of non-physiologic surfactants.

Providing a surfactant on the surfaces of the particles can reduce the tendency of the particles to agglomerate due to interactions such as electrostatic interactions, Van der Waals forces, and capillary action. The presence of the surfactant on the particle surface can provide increased surface rugosity (roughness), thereby improving aerosolization by reducing the surface area available for intimate particle-particle interaction.

Surfactants known in the art can be used including any naturally occurring surfactant. Other exemplary surfactants include diphosphatidyl glycerol (DPPG); hexadecanol; fatty alcohols (such as polyethylene glycol (PEG)); polyoxyethylene-9-lauryl ether; a surface active fatty acid (such as palmitic acid or oleic acid); sorbitan trioleate (Span 85); glycocholate; surfactin; a poloxamer; a sorbitan fatty acid ester (such as sorbitan trioleate); tyloxapol; and a phospholipid.

4.3 Cell Targeting Ligands

Particles (e.g., liposomes) can be targeted to receptors on APCs, for example, by placing ligands for cellular receptors of APCs on the surface of the particle (for example, mannosyl moieties or complement proteins such as C3d). In some embodiments of this type, the particulate parasite extracts additionally comprise a cell-targeting ligand on the surface of the particle. The cell-targeting ligand facilitates the delivery of the encapsulated parasite extract to an immune cell, for example, an APC. In some preferred embodiments, the immune cell is an APC, and even more preferably a dendritic cell and/or a macrophage. In some other preferred embodiments, the immune cell comprises a mannose receptor or a C-lectin type receptor on its cell surface.

In some embodiments, the cell-targeting ligand comprises a lipid anchor component, a linker component, and an oligosaccharide component.

In some embodiments, the oligosaccharide component comprises at least one mannosyl oligosaccharides. By way of an example, the mannosyl oligosaccharide may comprise 1, 2, 3, 4, 5 or 6 mannose residues.

The cell targeting the lipid anchor component suitably binds, attaches to or otherwise integrates with at least one layer of the lipid bilayer of the lipid vesicle. The lipid anchor may comprise, consist, or consist essentially of, at least one lipid or fatty acid chain thereof. Preferably the lipid is a C₄-C₂₀ lipid, or more preferably a C₁₂-C₁₈ lipid. By way of an illustrative example, the lipid may be a C₁₆ lipid, such as palmitate. The lipid may be saturated or unsaturated, although preferably saturated.

The targeting moiety may further comprise a linker component. Suitably, the linker or spacer is located or positioned between the lipid anchor and mannosyl oligosaccharide. The linker or spacer may comprise, consist, or consist essentially of one or more amino acids or peptides. Non-limiting examples of suitable amino acids include lysine and serine. In some embodiments, the linker or spacer may comprise polyethylene glycol. In certain embodiments, the spacer or linker may comprise one or more polyether compounds such as polyethylene glycol (PEG). The number of repeat units (O-CH2-CH2) may be 2-10. In some embodiments the linker component comprises six repeat units (O-CH2-CH2). Suitably, the linker or spacer may comprise two or more linked units comprising the polyether compounds such as polyethylene glycol (PEG).

In a preferred embodiment, the targeting moiety is a “mannosylated lipid core peptide” (MLCP). Typically, the MLCP may be of the general form of the cell targeting ligands described above. Particular MLCP embodiments designated F2-F5 are shown in Table 1. Embodiments designated F3 and F4 have been identified as particularly efficacious.

TABLE 1
EXEMPLARY CELL TARGETING LIGANDS

| CODE | OLIGOSACCHARIDE | LINKER | LIPID |
|---|---|---|---|
| F2 | mannose | Lys-Lys-Ser-Ser | C16 |
| F3 | mannose | Lys-Lys-Ser-Ser | 2 × C16 |
| F4 | mannose | (PEG₆)₂-Ser-Ser | C16 |
| F5 | (mannose)₄ | (Lys)₂-Lys-Ser-Ser | C16 |

In some particular embodiments wherein the parasite extracts are encapsulated in a lipid vesicle (e.g., a liposome), the cell-targeting ligand is at least partially embedded in the lipid bilayer of the liposome. Preferably, the cell-targeting is at least partially embedded in the outer layer of the lipid bilayer.

In some preferred embodiments, parasite extracts are encapsulated in a liposome with a cell targeting ligand at least partially embedded in the liposome lipid bilayer, wherein the cell targeting ligand is F3.

4.4 Adjuvants

In some embodiments the immunostimulatory compositions of the invention further comprise an adjuvant. Any adjuvant suitable for eliciting a Th1 immune response is applicable for use with the present invention. Some suitable adjuvants for use include Monophosphoryl Lipid A and/or its analogues, aluminium salts, oil-in-water emulsion, saponin, or a combination thereof.

In some preferred embodiments, the immunostimulatory compositions comprise a MPLA molecule or an MPLA analogue. MPLA has been shown to boost the immune system through the activation of the toll-like receptor 4 (TLR4), resulting in the production of proinflammatory cytokines and antigen-specific effector CD4+ and memory CD8+ T cells.

In some embodiments of this type, the adjuvant comprises Monophosphoryl Lipid A (MPLA; also referred to as GLA, and PHAD®) which has the following molecular structure:

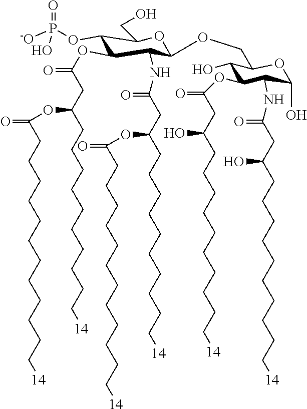

In some other embodiments of this type, the adjuvant comprises Monophosphoryl 3-Deacyl Lipid A, which is described in detail in U.S. Pat. No. 9,241,988 the contents of which is incorporated by reference. Specifically, Monophosphoryl 3-Deacyl Lipid A, which the following molecular structure:

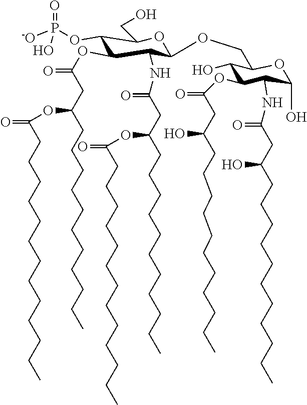

In some other embodiments of this type, the adjuvant comprises Monophosphoryl Hexa-acyl Lipid A, 3-Deacyl, with the following molecular structure:

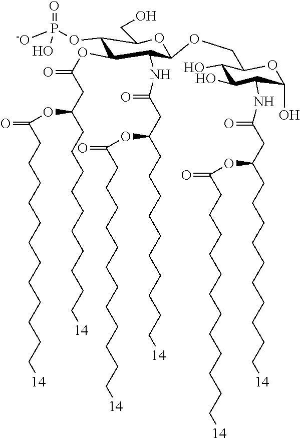

In some other embodiments of this type, the adjuvant comprises Monophosphoryl Lipid A-504, with the following molecular structure:

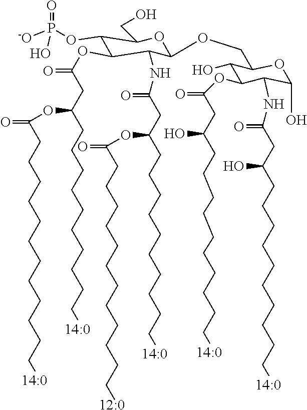

In some other embodiments, the adjuvant comprises an MPLA derived from the hydrolysis endotoxin lipopolysaccharide (LPS) isolated from Salmonella minnesota R595. Although the hydrolysis reaction results in several species, the predominant species has the following molecular structure:

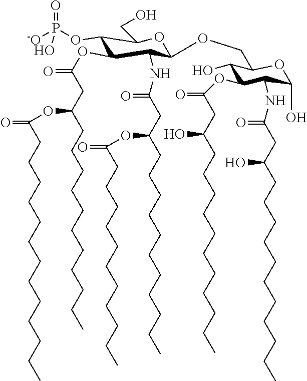

In some other embodiments, the adjuvant comprises trehalose dibehenate (TDB), with the following molecular structure:

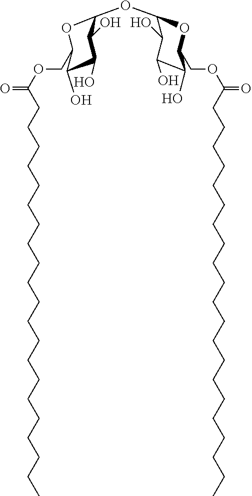

In some other embodiments, the adjuvant comprises dimethyldioctadecylammonium (DDA), with the following molecular structure:

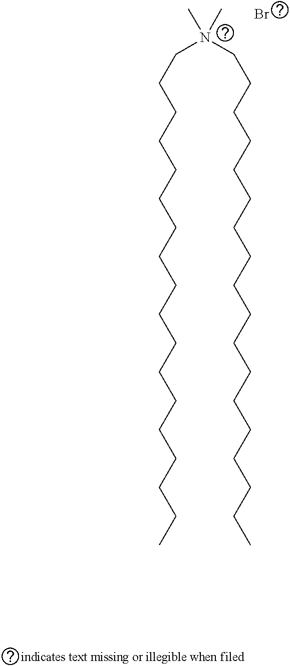

The adjuvants described above may be associated with the particle, so that the oligosaccharide is displayed on the surface of the particle. In lipid vesicle embodiments, the lipid portion of the adjuvant may be at least partially embedded in the lipid membrane.

Alternatively, the adjuvant may be encapsulated within the particle, or external to the particle. In embodiments wherein the adjuvant is external to the particle the adjuvant may be co-administered with the parasite extract.

In some embodiments, the particle is a liposome and the adjuvant is at least partially embedded in the liposome lipid membrane. In some embodiments of this type, the adjuvant is MPLA (PHAD). In some preferred embodiments of this type, the liposome also comprises a cell targeting ligand (e.g., F3). In other preferred embodiments, the liposome does not comprise a cell targeting ligand.

### 5. Methods of Manufacture

In some embodiments, the malaria parasites are inactivated while in the pRBC and are then subsequently extracted. As hereinbefore described, the parasite extract is treated to remove RBC components such as cell membranes, membrane proteins, and soluble proteins. In some embodiments of this type, greater than around 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% of the total RBC components are removed from the parasite extract.

In some embodiments, the pRBC are lysed to release the parasites. Such conditions must be sufficient to lyse the membrane of the RBC cells, but not sufficient to significantly lyse the parasite membranes (i.e., leave the parasite cells in-tact). For example, in some preferred embodiments the RBC are lysed by exposing the cells to a chemical lysis buffer. One such suitable lysis buffer that is particularly suitable for freeing parasites from RBCs is saponin. In some embodiments, saponin is used at a concentration of between about 0.05% and about 3% saponin, however, any suitable concentration of the lysis buffer can be used. In some preferred embodiments, the chemical lysis buffer is a 0.15% saponin in phosphate buffered saline (PBS).

In some embodiments, it can be advantageous to remove the soluble RBC components from the parasite preparation. For example, the inclusion of soluble proteinaceous RBC molecules prohibits the accurate estimation of parasite antigenic protein concentration, in light of the contribution of the soluble RBC matter. In order to remove the soluble RBC components (e.g., haemoglobin) from the parasite preparations, the insoluble fraction (containing the whole parasites and are RBC membranes) is harvested (e.g., separated) from the soluble matter. Any suitable separation technique can be used for this purpose. In some preferred embodiments, centrifugation under suitable conditions is used to pellet the insoluble matter. Once the separation has occurred the supernatant can be removed and the pellet resuspended in buffer (e.g., phosphate buffered saline (PBS)). In some preferred embodiments this step is performed multiple times (e.g., 2, 3, 4, 5, or more times) to ensure that substantially all of the soluble RBC components are removed.

The parasite extract may then be exposed to conditions sufficient to lyse the parasite membranes, in order to release the soluble parasite antigens. This may be achieved by any suitable method, including freeze-thaw cycles. Typically, the freezing occurs at about −80° C., however, any temperature suitable to cause cells to swell and break (for example, due to the formation of ice crystals) is equally applicable in the methods of the present invention. In some preferred embodiments, however, the method of lysing parasite membranes does not comprise detergent (e.g., Triton X-100 detergent), due at least in part to its known toxicity and ability to interfere with lipid membranes.

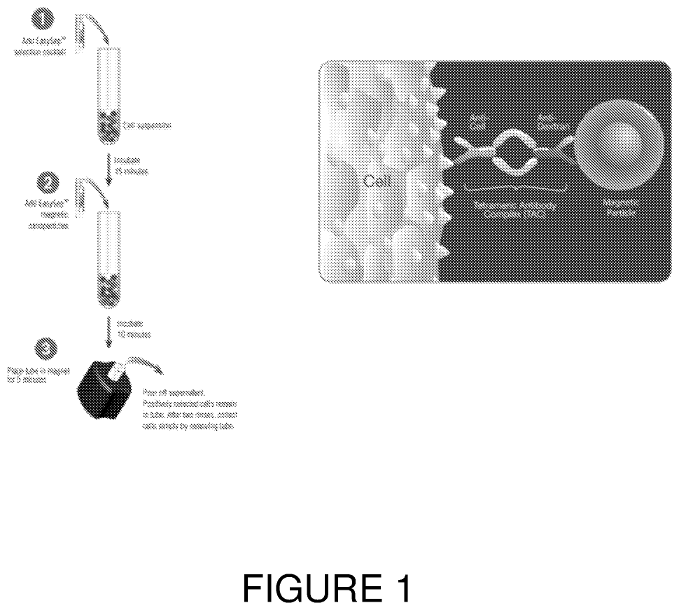

In some embodiments, the insoluble fraction (comprising components such as RBC membranes and parasite membranes) is separated from the soluble fraction (comprising soluble parasite antigens) using any suitable separation method. For example, the soluble parasite antigens may be harvested by centrifugation, and the supernatant retained. In some of the same embodiments and some other embodiments, the RBC membranes are subsequently removed from the preparation by a process that includes immuno-affinity depletion. This process may include using an antibody specific for an RBC membrane molecule to form a complex with the RBC membranes. Suitable RBC membrane molecules of this type include a glycophorin or any other RBC membrane protein not expressed by the parasite (such as Rh antigens, band 3 or aquaporin proteins, or sugars such as blood group A and/or B antigens). In embodiments of this type, the antibody specific for the RBC membrane protein (e.g., glycophorin A) is coupled to a substrate (e.g., a particle or bead) which facilitates separation of the RBC membranes comprising the membrane protein from the treated malaria parasites. In one particular embodiment, the particle or bead is a magnetic particle which enables magnetic separation of the RBC membranes from the treated malaria parasites. A particular non-limiting example is the EASYSEP Human Glycophorin Depletion system available from StemCell Technologies (schematically depicted in FIG. 1). In some of the same embodiments and some other embodiments, the soluble parasite antigen fraction is obtained through filtration. Any suitable filtration techniques with a pore size sufficient to retain the insoluble components (e.g., broken membranes) is applicable for use to product the parasite extracts of the invention.

In some embodiments, a combination of two or more techniques to separate the soluble parasite antigen from the insoluble matter may be used.

In some embodiments, a single separation step is performed, using immune-affinity depletion. As such, the parasite extract may be substantially free of RBC components (such as RBC membranes and RBC soluble components).

In some preferred embodiments, the preparation of the parasite extracts is performed at a temperature between about 4° C. and about 40° C. The soluble parasite fraction typically comprises many antigenic molecules, some of which are not heat-stable. Therefore, in an effort to retain as many antigenic molecules as possible, including those that denature upon increased temperatures (i.e., above 40° C.) the extraction process is preferably performed at 37° C. or below.

5.1 Preparation of Liposomes

Liposomes can be produced by standard methods such as those reported by Kim et al., (1983, Biochim. Biophys. Acta 728, 339-348); Liu et al., (1992, Biochim. Biophys. Acta 1104, 95-101); Lee et al., (1992, Biochim. Biophys. Acta. 1103, 185-197); Brey et al., (U.S. Patent Applicant Pub. 20020041861), Hass et al., (U.S. Patent Application Publication No. 2005/0232984); Kisak et al., (U.S. Patent Application Publication No. 2005/0260260) and Smyth-Templeton et al., (U.S. Patent Application Publication No. 2006/0204566). Additionally, reference may be made to Copeland et al., (2005, Immunol. Cell Biol. 83: 95-105) who review lipid based particulate formulations for the delivery of antigen, and to Bramwell et al., (2005, Crit Rev Ther Drug Carrier Syst. 22(2): 151-214; 2006, J Pharm Pharmacol. 58(6):717-728) who review particulate delivery systems for vaccines, including methods for the preparation of protein-loaded liposomes. Many liposome formulations using a variety of different lipid components have been used in various in vitro cell culture and animal experiments. Parameters have been identified that determine liposomal properties and are reported in the literature, for example, by Lee et al., (1992, Biochim. Biophys. Acta. 1103, 185-197); Liu et al., (1992, Biochim. Biophys. Acta. 1104, 95-101); and Wang et al., (1989, Biochem. 28, 9508-951).

Briefly, the lipids of choice (and any organic-soluble bioactive), dissolved in an organic solvent, are mixed and dried onto the bottom of a glass tube under vacuum. The lipid film is rehydrated using an aqueous buffered solution containing any water-soluble parasite extracts to be encapsulated by gentle swirling. The hydrated lipid vesicles can then be further processed by extrusion, submitted to a series of freeze-thawing cycles or dehydrated and then rehydrated to promote encapsulation of antigenic preparations. Liposomes can then be washed by centrifugation or loaded onto a size-exclusion column to remove unentrapped bioactive from the liposome formulation and stored at 4° C. The basic method for liposome preparation is described in more detail in Thierry et al., (1992, Nuc. Acids Res. 20:5691-5698).

In some embodiments, cell-targeting ligands and/or lipid adjuvants can be at least partially encompassed in the lipid bilayer of the liposome. In such embodiments, an appropriate amount of the molecule can be included in the liposome preparation. The exact amount of cell-targeting ligand and/or adjuvant will be independently dependent on a range of properties, including but not limited to the affinity of the molecule to bind its target, the concentration of the target, the half-life of the molecule, etc.

### 6. Methods of Use

The compositions of the invention may be used for stimulating an immune response to a parasite antigen in a subject that is immunologically naïve to the parasite antigen or that has previously raised an immune response to that parasite antigen. Thus, the present invention also extends to methods for enhancing an immune response in a subject by administering to the subject the compositions or vaccines of the invention. In some embodiments, the immune response is a cell-mediated immune response (e.g., a T-cell mediated response, which desirably includes CD4⁺ T cells). Accordingly, one aspect of the invention relates to eliciting an immune response to a parasite antigen in a mammal, by administering to the mammal a composition comprising a parasite extract, wherein the parasite extract substantially free of RBC components (such as membranes and soluble components) in a particle, to thereby elicit an immune response to the malaria parasite in the mammal. In some embodiments, the parasite extract is substantially free of insoluble parasite components.

Also encapsulated by the present invention is a method for treatment and/or prophylaxis of a parasitic disease, comprising administering to a subject in need of such treatment an effective amount of an immunostimulating composition, as broadly described above. In certain embodiments, the parasitic disease is selected from the group comprising malaria, babesiosis, cryptosporidiosis, cyclosporiasis, cystoisosporiasis, and toxoplasmosis.

In some embodiments of this type, invention provides a method of treating or preventing malaria in a mammal, by administering to the mammal an immunogenic agent comprising a malaria parasite extract substantially free of RBC components (e.g., RBC membranes and soluble RBC components) in a particle (e.g., lipid vesicle), to thereby treat or prevent malaria in the mammal. In some embodiments, the parasite extract is substantially free of insoluble parasite components.

Still yet other aspects of the invention relate to immunizing a mammal against a parasitic disease, by administering to the subject an immunostimulatory composition comprising a parasite extract substantially free of RBC components (e.g., RBC membranes and soluble RBC components) in a particle (e.g., lipid vesicle), to thereby immunize the subject against the parasitic disease. In some embodiments, the parasite extract is substantially free of insoluble parasite components.

In other embodiments, the composition of the invention could also be used for generating large numbers of CD8⁺ or CD4⁺ cytotoxic T cells (CTLs), for adoptive transfer to immunodeficient individuals who are unable to mount normal immune responses. For example, antigen-specific CD8⁺ CTL can be adoptively transferred for therapeutic purposes in individuals afflicted with a malaria infection (see, Cavacini, et al., 1986).

The effectiveness of the immunization may be assessed using any suitable technique. For example, CTL lysis assays may be employed using stimulated splenocytes or peripheral blood mononuclear cells (PBMC) on peptide coated or recombinant virus infected cells using ⁵¹Cr or Alamar Blue™ labelled target cells. Such assays can be performed using for example primate, mouse or human cells (Allen et al., J Immunol, 2000, 164(9): 4968-4978 also Woodberry et al., infra). Alternatively, the efficacy of the immunization may be monitored using one or more techniques including, but not limited to, HLA class I tetramer staining—of both fresh and stimulated PBMCs (see for example Allen et al., supra), proliferation assays (Allen et al., supra), ELISPOT assays and intracellular IFN-γ staining (Allen et al., supra), ELISA Assays—for linear B cell responses; and Western blots of cell sample expressing the synthetic polynucleotides.

6.1 Prime-Boost Regimens

Certain embodiments of the present invention involve the administration of pharmaceutical compositions in multiple separate doses. Accordingly, the methods for the prevention (i.e., vaccination) and treatment of infection described herein encompass the administration of multiple separated doses to a subject, for example, over a defined period of time. Accordingly, the methods for the prevention (i.e., vaccination) and treatment of infection disclosed herein include administering a priming dose of a pharmaceutical composition of the present invention. The priming dose may be followed by a booster dose. The booster may be for the purpose of re-vaccination. In various embodiments, the pharmaceutical composition or vaccine is administered at least once, twice, three times or more.

The methods of the invention may comprise administering a priming composition that comprises the parasite soluble extract as broadly defined above and elsewhere herein, wherein the parasite soluble extract stimulates or otherwise enhances an immune response to a parasite antigen in a subject, and subsequently administering a later booster composition of the parasite soluble extract as broadly defined above and elsewhere herein. Alternatively, the booster composition may be a different composition to the priming composition.

In some embodiments, the booster composition may be administered at least 7, 14, 21 or 28 days, at least 1, 2, 3, 4, 5, or 6 months, or at least 1, 2, 3, 4, or 5 years after the priming composition. The priming and booster compositions may be administered by the same or different routes. For example, the priming and booster doses may both be administered—subcutaneously, intramuscularly, intravenously, or intraperitoneally. Alternatively, the priming dose may be administered locally (e.g., mucosally, such as intranasally) to induce mucosal antigen-specific immune cells, and the booster dose administered subcutaneously, intramuscularly, or intravenously to induce systemic antigen-specific immune cells. In some preferred embodiments, the booster dose is administered intramuscularly.

The immunogenic composition and method of prevention or treatment of malaria may elicit an immune response that is characterized as a CD4⁺ T cell-mediated response, including solely CD4⁺ T cell-mediated responses and mixed CD4⁺ and CD8⁺ T cell-mediated responses, expression of cytokines such as IFN-γ and typically with little or no antibody response. Suitably, following administration to a mammal, the immunogenic agent enhances, optimizes or otherwise promotes activation of APCs including uptake of malaria parasite antigens by APCs and their subsequent maturation. Activated APCs may be characterized by the expression of MHC-II and/or costimulatory molecules such as CD80 and CD86. Suitably, expression and secretion of IFN-γ. Preferably, the immunogenic composition and/or method immunize the mammal to prevent, inhibit or otherwise protect the mammal against subsequent malaria infection.

In some embodiments, either one of the priming composition or the booster composition comprises low dose live malaria parasites and an anti-malarial drug. For example, in some embodiments the priming composition comprises the parasite soluble extract as broadly defined above and elsewhere herein, and one or more booster compositions comprise a low dose of malaria parasites (e.g., Plasmodium falciparum) and an anti-malaria drug. In some alternative embodiments, the priming composition comprises a low dose of malaria parasites (e.g., Plasmodium falciparum) and an anti-malaria drug, and one or more booster composition comprise the parasite soluble extract as broadly defined above and elsewhere herein.

By a “low dose” of malaria parasites generally refers to a dose that is not substantial enough to result in a malaria infection in a mammal (e.g., human), but is sufficient for the mammal (e.g., human) to generate an immune response to the administered malaria parasites. Typically, such low dose can be considered to be between about 10⁴ malaria parasites per dose and about 10⁸ malaria parasites per dose. Preferably, the low dose of malaria parasites (e.g., Plasmodium falciparum) comprises between about 10⁵ parasites per dose and about 10⁸ malaria parasites per dose.

In some embodiments of this type, any anti-malarial drug may be suitable for this use, including combination therapies of two or more active agents. Suitable anti-malaria drugs (some of which combine active agents) include, but are not limited to, the group comprising: atovaquone+proguanil hydrochloride (MALARONE, Galaxosmithkline); tafenoquine (ARAKODA, Glaxosmithkline); artemether+lumefantrine (COARTEM, Novartis International AG); piperaquine tetraphosphate+artenimol (EURARTESIM, Sigma-Tau Pharmaceuticals, Inc.); pyronaridine+artesunate (PYRAMAX, Shin Poong Pharmaceutical Co. Ltd.); artesunate+amodiaquine (ASAQ WINTHROP, Sanofi SA); artesunate+mefloquine (ASMQ, Cipla Limited); amodiaquine+sulfadoxine-primethamine (SPAQ-CO/SUPYRA, Guilin Pharmaceutical Co., Ltd); artesunate (ARTESUN, Guilin Pharmaceutical); azithromycin; artemether; artesunate; dihyrdroartemisinin; lumefantrine; amodiaquine; mefloquine; piperaquine; chloroquine; doxycycline; primaquine; sulfadoxine-pyrimethamine; OZ439/PQP (Sanofi); OZ439/FQ (Sanofi); KAE609 (Novartis); DSM265 (NIH/Takeda); and MK-4815 (Merk & Co., Inc).

As hereinbefore described, the invention provides immunogenic agents and/or methods of preventing or treating a parasitic disease or an associated disease, disorder or condition in a mammal. In some embodiments, the subject is a human.

In certain aspects and embodiments, the immunogenic agent may be administered in the form of a formulation together with an acceptable carrier, diluent or excipient.

By “acceptable carrier, diluent or excipient” is meant a solid or liquid filler, diluent or encapsulating substance that may be safely used in systemic administration. Depending upon the particular route of administration, a variety of carriers, diluent and excipients well known in the art may be used. These may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids (such as acetates, propionates and malonates), water and pyrogen-free water.

A useful reference describing acceptable carriers, diluents and excipients is Remington's Pharmaceutical Sciences (Mack Publishing Co. N.J. USA, 1991) which is incorporated herein by reference.

In certain aspects the liposomal and targeting formulation can be co-administered with additional adjuvants, such as including aluminium salts, emulsions (e.g., oil-in-water emulsion such as MF59 and AS03 or water-in-oil emulsion such as Montanide ISA-51 and Montanide ISA-720), monophosphoryl lipid A analogues, saponins (e.g., Quil-A and fractions thereof, including QS21), or combinations of these.

Any safe route of administration may be employed, including intranasal, oral, rectal, parenteral, sublingual, buccal, intravenous, intraarticular, intramuscular, intradermal, subcutaneous, inhalational, intraocular, intraperitoneal, intracerebroventricular, topical, mucosal and transdermal administration, although without limitation thereto.

Dosage forms include tablets, dispersions, suspensions, injections, solutions, syrups, troches, capsules, nasal sprays, suppositories, aerosols, transdermal patches and the like. These dosage forms may also include injecting or implanting controlled releasing devices designed specifically for this purpose or other forms of implants modified to act additionally in this fashion. Controlled release may be effected by coating with hydrophobic polymers including acrylic resins, waxes, higher aliphatic alcohols, polylactic and polyglycolic acids and certain cellulose derivatives such as hydroxypropylmethyl cellulose.

Formulations may be presented as discrete units such as capsules, sachets, functional foods/feeds or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such formulations may be prepared by any of the methods of pharmacy but all methods include the step of bringing into association one or more agents as described above with the carrier which constitutes one or more necessary ingredients. In general, the formulations 20 are prepared by uniformly and intimately admixing the agents of the invention with liquid carriers or finely divided solid carriers or both, and then, if necessary, shaping the product into the desired presentation.

The above formulations may be administered in a manner compatible with the dosage formulation, and in such amount as effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner. Typical dosages for administration to a mammal may be about 10¹ to about 10¹⁰ malaria parasites per dose of immunogenic agent, including dosages such as 5×10⁹, 5×10⁸, 5×10⁷, 5×10⁶, 5×10⁵, 5×10⁴, 5×10³, 5×10², or 5×10¹ blood-stage malaria parasites or any dosage or dosage range between these.

### 7. Pharmaceutical Formulations

In accordance with the present invention, the parasite extracts are useful in compositions and methods for stimulating an immune response to one or more parasite antigens. These compositions are useful, therefore, for treating or preventing a parasitic disease, for example, malaria.

The compositions of the invention are, therefore, useful for stimulating an immune response to a parasite antigen in a subject, which comprises administering to the patient a pharmaceutical composition comprising one or more parasite extracts as described above and/or elsewhere herein. The pharmaceutical composition may comprise a pharmaceutically acceptable carrier or diluent. In some embodiments, the compositions are administered to individuals who have been diagnosed with a parasitic disease. In other embodiments, the compositions are administered to at-risk individuals who are identified as being at risk of being exposed to a parasite (e.g., where a subject is travelling to a location where an apicomplexan parasite is known to be prevalent).

Pharmaceutical compositions suitable for use in the present invention include compositions wherein the parasite antigens are contained in an effective amount to achieve their intended purpose (i.e., to stimulate an immune response in the subject). The dose of active compound(s) administered to a patient should be sufficient to achieve a beneficial response in the patient over time such as eliciting or enhancing an immune response, which is suitably associated with a condition (e.g., malaria). The quantity or dose frequency of the pharmaceutically active compounds(s) to be administered may depend on the subject to be treated inclusive of the age, sex, weight and general health condition thereof. In this regard, precise amounts of the active compound(s) for administration will depend on the judgement of the practitioner. In determining the effective amount of the active compound(s) to be administered in the treatment or prophylaxis of the parasitic disease, the practitioner may evaluate inflammation, proinflammatory cytokine levels, lymphocyte proliferation, cytolytic T lymphocyte activity and regulatory T lymphocyte function. In any event, those of skill in the art may readily determine suitable dosages of the extract.

Accordingly, the bioactive agents are administered to a subject to be treated in a manner compatible with the dosage formulation, and in an amount that will be prophylactically and/or therapeutically effective. The amount of the composition to be delivered, generally in the range of from about 0.01 μg/kg to about 100 μg/kg of bioactive molecule (e.g., parasite antigen etc.) per dose, depends on the subject to be treated. In some embodiments, and dependent on the intended mode of administration, the antigen-containing compositions will generally contain about 0.1% to 90%, about 0.5% to 50%, or about 1% to about 25%, by weight antigen, the remainder being suitable pharmaceutical carriers, diluents, adjuvants etc. The dosage of the inhibitor can depend on a variety of factors, such as mode of administration, the species of the affected subject, age and/or individual condition. In other embodiments, and dependent on the intended mode of administration.

Depending on the specific condition being treated, the particles may be formulated and administered systemically, topically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, transcutaneous, intradermal, intramedullary delivery (e.g., injection), as well as intrathecal, direct intraventricular, intravenous, intraperitoneal, intranasal, or intraocular delivery (e.g., injection). For injection, the parasite extracts of the invention may be formulated in aqueous solutions, suitably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

The compositions of the present invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives, and hydrophilic beeswax derivatives.

Alternatively, the parasite extracts of the present invention can be formulated readily using pharmaceutically acceptable carriers well known in the art into dosages suitable for oral administration, which is also contemplated for the practice of the present invention. Such carriers enable the extracts of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical formulations for parenteral administration include aqueous solutions of the particles in water-soluble form. Additionally, suspensions of the bioactive agents may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances that increase the viscosity of the suspension, such as sodium carboxymethyl cellulose, sorbitol, or dextran. Optionally, the suspension may also contain suitable stabilisers or agents that increase the solubility of the compounds to allow for the preparation of highly concentrated solutions.

Pharmaceutical preparations for oral use can be obtained by combining the bioactive agents with solid excipients and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy, but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of particle doses.

Pharmaceuticals which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The parasite extracts of the present invention may be administered over a period of hours, days, weeks, or months, depending on several factors, including the severity of the condition being treated, whether a recurrence of the condition is considered likely, etc.

The administration may be constant, e.g., constant infusion over a period of hours, days, weeks, months, etc. Alternatively, the administration may be intermittent, e.g., bioactive agents may be administered once a day over a period of days, once an hour over a period of hours, or any other such schedule as deemed suitable.

The parasite extracts of the present invention may also be administered to the respiratory tract as a nasal or pulmonary inhalation aerosol or solution for a nebulizer, or as a microfine powder for insufflation, alone or in combination with an inert carrier such as lactose, or with other pharmaceutically acceptable excipients. In some particulate embodiments of the present invention, the particles of a formulation may advantageously have diameters of less than about 50 μm, suitably less than about 10 μm.

In some particulate embodiments, the parasite extracts are administered for active uptake by cells, for example by phagocytosis, as described for example in U.S. Pat. No. 5,783,567. In some embodiments, phagocytosis by these cells may be improved by maintaining a particle size typically below about 20 μm, and preferably below about 11 μm.

In specific particulate embodiments, parasite extracts in particulate form are delivered directly into the bloodstream (i.e., by intravenous or intra-arterial injection or infusion) if uptake by the phagocytic cells of the reticuloendothelial system (RES), including liver and spleen, is desired. Alternatively, and in some preferred embodiments, one can target, via subcutaneous injection, take-up by the phagocytic cells of the draining lymph nodes. The particles can also be introduced intradermally (i.e., to the APCs of the skin, such as dendritic cells and Langerhans cells) for example using ballistic or microneedle delivery. Illustrative particle-mediated delivery techniques include explosive, electric or gaseous discharge delivery to propel carrier particles toward target cells as described, for example, in U.S. Pat. Nos. 4,945,050, 5,120,657, 5,149,655 and 5,630,796. Non-limiting examples of microneedle delivery are disclosed in International PCT Publication Nos. WO 2005/069736 and WO 2005/072630 and U.S. Pat. Nos. 6,503,231 and 5,457,041.

In other specific particulate embodiments, the route of particle delivery is via the gastrointestinal tract (e.g., orally). Alternatively, the particles can be introduced into organs such as the lung (e.g., by inhalation of powdered microparticles or of a nebulized or aerosolized solution containing the microparticles), where the particles are picked up by the alveolar macrophages, or may be administered intranasally or buccally. Once a phagocytic cell phagocytoses the particle, the parasite extracts are released into the interior of the cell.

### 8. Kits

The present invention also provides kits comprising an immunostimulatory compositions as broadly described above and elsewhere herein. Such kits may additionally comprise alternative immunogenic agents for concurrent use with the immunostimulatory compositions of the invention.

In some embodiments, in addition to the immunostimulatory compositions of the present invention the kit may include suitable components for performing the prime-boost regiments described above. For example, the kit may include a priming dose of a parasite extract.

The kit may comprise additional components to assist in performing the methods of the present invention such as, for example, administration device(s), buffer(s), and/or diluent(s). The kits may include containers for housing the various components and instructions for using the kit components in the methods of the present invention.

In order that the invention may be readily understood and put into practical effect, particular preferred embodiments will now be described by way of the following non-limiting examples.

### EXAMPLES

### Example 1

### Construction of Liposomes and Removal of Red Cell Membranes

Liposomes comprising parasitised red blood cells (pRBC) were formulated using cationic lipids with (mannose-Lys-Lys-Lys-Ser-Ser)-(C₁₆)₂, referred to as “F3”, anchored into the liposome membrane by its two palmitic acid molecules. Parasite antigen was incorporated into the liposomes during hydration of the lipids. Liposomes containing parasite antigen (without the removal of red cell membranes) were constructed such that each mouse received approximately 1×10⁷ parasite equivalents.

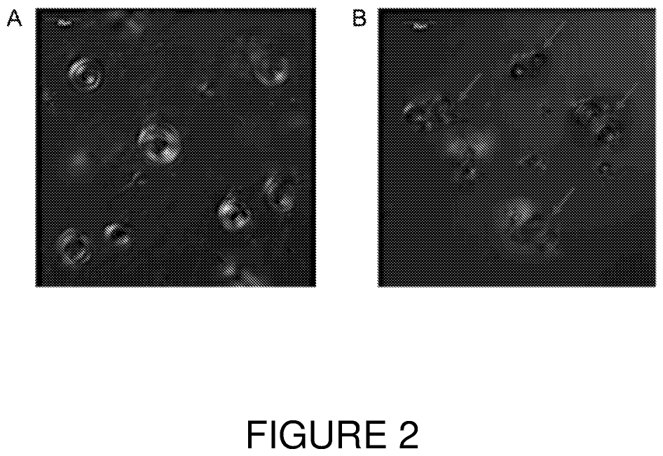

Liposomes were also constructed using parasite antigen from which human red cell membranes were removed using magnetic beads and antibodies to Glycophorin A (GlyA). GlyA is a membrane-spanning sialo-glycoprotein of human red blood cells and used here as a surrogate for the presence of human red cell membranes. It has been implicated in alloimmunization and autoimmune haemolytic anaemia (see, Brain et al., 2002). In the experiments described below, we used FACS to determine the median fluorescent intensity (MFI) of particulate parasite antigen following staining with antibodies to GlyA (to determine the presence of red cell membranes) and Hoechst to measure parasite DNA (as a surrogate for parasite antigen) (see, FIG. 2).

After establishing the methodology, two preparations were made. It was estimated there to be 86% and 92% depletion of red cell membranes from preparations 1 and 2, respectively.

The amount of DNA remaining varied (see, Table 2). The amount of post-red cell membrane depleted parasite material to inoculate was calculated based on the starting concentration of parasites, allowing for an arbitrary loss of 10% as a result of the depletion processes, and a desired dose of 10⁷ parasite equivalents per inoculum. However, using back calculations based on the actual MFI of Hoechst staining of the parasite material, we found that liposomes made from preparation 1 contained 7.3×10⁶ parasite equivalents per dose and liposomes made from preparation 2 contained 1.7=10⁷ parasite equivalents per dose (see, Table 2).

TABLE 2
ESTIMATION OF RED BLOOD
CELL MEMBRANE DEPLETION

| | Preparation 1¹ | Preparation 2² |
|---|---|---|
| Pre-magnetic purification (MFI) | 1082 | 2258 |
| Post-magnetic purification (MFI) | 151 | 179 |
| % Depletion | 86 | 92 |
| Red cell membrane equivalents post | 1.4 × 10⁶ | 8 × 10⁵ |
| purification per dose | | |

Confirmation of parasite DNA

| | | |
|---|---|---|
| Pre-magnetic purification (MFI) | 4215 | 6972 |
| Post-magnetic purification (MFI) | 3126 | 11963 |
| % DNA loss | 25.8 | −71.6 |
| DNA parasite equivalents/dose post | 7.42 × 10⁶ | 1.716 × 10⁷ |
| purification per dose | | |

¹Preparation 1 was used in experiments in which the immunogenicity of liposomes containing parasite antigens with or without red cell membrane depletion was evaluated.
²Preparation 2 was used in experiments in which the immunogenicity of fresh versus lyophilized liposomes was examined.

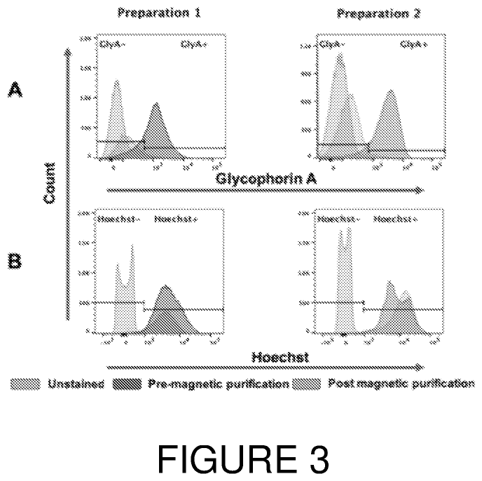

Analysis of particle size, measured as volume-weighted diameter, indicated that freshly prepared and lyophilized liposomes were approximately 32.2 and 65.3 μm in diameter with width of particle size distribution (span) values of 2.3 and 2.2 respectively. These data indicated that lyophilized liposomes were twice the size of their freshly prepared counterparts. Confocal microscopy showed that re-hydrated lyophilized liposomes tended to form aggregates as shown in FIG. 3, and hence the larger particle size distribution.

### Materials and Methods

Mice

Inbred Bagg Albino (BALB/c) (H-2d) female mice aged 4-6 weeks were purchased from the Animal Resource Centre (ARC) (Canning Vale, Western Australia). All animals were housed under special pathogen free, Physical Containment level 2 (PC2) conditions in the Griffith University Institute for Glycomics animal facility.

All animal procedures were performed in accordance with the Australian Code for the Care and Use of Animals for Scientific Purposes 8th edition (2013) and approved by Griffith University Animal Ethics Committee under ethics approval numbers GLY/07/16/AEC, and GLY/15/16/AEC.

Human and Rodent Malaria Parasites

All liposomes were formulated using a P. falciparum 7G8 laboratory line (see, Stanisic et al., 2015). Clinical P. falciparum isolate UGMCB-0009 from Uganda (see, Ssemaganda et al., 2018), P. knowlesi A1H.1 (see, Moon et al., 2013), and P. yoelii 17X were used to examine the breadth of strain/species-specific immune responses following immunization.

Culture of P. falciparum and P. knowlesi Parasitized Red Blood Cells

Frozen vials containing P. falciparum or P. knowlesi parasitized red blood cells (pRBCs) in glycerolyte solution (Baxter) were thawed and cultured as described previously (see, Stanisic et al., 2015, Ssemaganda et al., 2018; and Arnold et al., 2016). Parasite cultures were monitored regularly by thin blood films. Thin blood films were stained with Giemsa and observed by microscopy to ascertain the parasitaemaia and life-cycle stage. Mature parasite forms (trophozoites and schizonts), purified from parasite cultures (as described below), were used for preparation of whole parasite antigen in liposomes (P. falciparum) and for in vitro stimulation (P. falciparum and P. knowlesi) in splenocyte proliferation assays.

Purification of P. falciparum and P. knowlesi Trophozoi

To reduce the number of uninfected red blood cells, present in the parasite culture for the vaccine, trophozoite/schizont-stage pRBC were purified from culture by magnetic separation using CS columns (Miltenyi Biotec) on the MACS Vario system (Miltenyi Biotec). The pRBC preparations at >98% purity—were frozen (−80° C.) at a desired concentration until required for liposome formulation or for use in in vitro stimulation experiments.

Depletion of Red Cell Membranes from Purified P. falciparum Preparations

Red cell membranes were depleted from the purified P. falciparum preparations using the EasySep Human Glycophorin A Depletion Kit (STEMCELL Technologies) according to the manufacturer's instructions. Briefly, the pRBC suspension at a parasite equivalent concentration required for use in vaccine formulation, was subjected to six freeze-thaw cycles and resuspended in Phosphate Buffered Saline (PBS). The EasySep Human Glycophorin A depletion cocktail (STEMCELL Technologies) was added to the pRBC suspension and incubated at room temperature for 15 min. Next, EasySep Magnetic positive selection nanoparticles (STEMCELL Technologies) were added and incubated for 10 minutes. Immunomagnetic separation was then performed by placing the tube containing the pRBC cell suspension into a magnetic field (STEMCELL Technologies) and incubating for an additional 10 min at room temperature. The enriched parasite antigen suspension was decanted in one continuous motion into a new tube and aliquoted at the desired parasite equivalent, approximately 10⁷ pRBCs required per mouse as the vaccine immunizing dose.

These parasite antigen extracts were used in formulating liposomes containing red-cell membrane depleted antigens. To confirm the removal of red cell membranes and the presence of parasite antigen following magnetic purification, 200 μL of the parasite extract was centrifuged for 10 min at 16,000 g. The pellet was subsequently stained with anti-Glycophorin A FITC (Clone H1264, BioLegend) and bisbenzimide Hoechst (Sigma-Aldrich) for 20 min at room temperature, then washed in MACS buffer. Samples were acquired on a BD LSR Fortessa (BD Biosciences) and data analysed using FlowJo software version 10.0 (Tree Star).

Preparation of P. falciparum Parasite Antigen without Depletion of Red Cell Membrane Antigen

Plasmodium falciparum-infected red cells at the desired concentration based on parasite equivalence prior to red cell membrane depletion were subjected to six freeze-thaw cycles and resuspended in an appropriate volume of PBS ready for use in the preparation of the liposomes.

Formulation of Liposomes Containing P. falciparum Antigens and Immunizations

Liposomes were prepared using the thin film hydration method as described previously (see, Giddam et al., 2016). Liposomes consisted of 1,2-dipalmitoylsn-glycero-3-phosphocholine (DPPC, Avanti polar lipids), dimethyldioctadecylammonium bromide (DDAB, Sigma-Aldrich) and cholesterol taken in the ratio of 5:2:1. Additionally, 10 μg of the mannosylated lipid core peptide, referred to as ‘F3’ hereon in, comprised of a lysine-serine-serine (KSS) spacer and 2-amino-D, L-hexadecanoic acid, was added per dose of vaccine formulation. F3 was synthesized as previously described (see, Giddam et al., 2016). F3, DPPC, DDAB and cholesterol were dissolved in a methanol/chloroform (1:9 v/v) solvent mixture which was then evaporated under vacuum (Heidolph) forming a thin lipid film at the base of the flask. Hydration was performed at 50° C. using parasite antigen solution of PBS for empty liposomes.

### Example 2

### Evaluation of the Induction of Anti-Human Red Blood Cell Antibodies in Mice

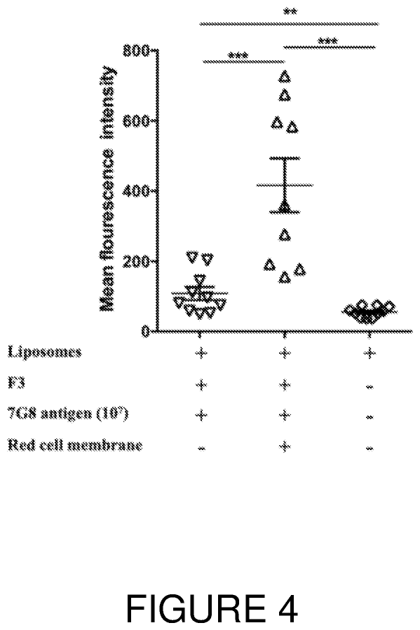

It was then assessed whether immunization of mice with F3 liposomes containing red cell membrane-depleted P. falciparum antigens resulted in the induction of anti-human red blood cell (hRBC) antibodies. For these experiments, parasite antigen from preparation 1 (see, Table 2) was used to construct the liposomes from which red cell membranes were removed. Flow cytometric analysis revealed a significant reduction in anti-hRBC antibodies in mice immunized with freshly prepared red cell membrane-depleted liposomes compared to mice that received liposomes made without depletion of red cell membranes (P<0.0001) (see, FIG. 4). Although significantly reduced, there was still a small but significant increase in the MFI in mice vaccinated with red-cell membrane-depleted liposomes compared to mice vaccinated with ‘empty’ liposomes (P<0.002) (see, FIG. 4). This was likely due to the small amount of residual red cell membrane material present (Table 2). Nevertheless, these data support the use of immune-depletion as a method to reduce the induction of alloantibodies to human red cells.

### Materials and Methods

Measuring Induction of Peripheral Blood Antigen-Experienced T Cells Following Vaccination

Circulating antigen-experienced T cells were assessed following previously published methods [16]. At least 10,000 CD3⁺ events were acquired on a BD LSR Fortessa (BD Biosciences) and the data were analyzed using FlowJo software version 10.0 (Tree Star).

Anti-Human Red Cell Antibody Binding Assay

To examine the induction of anti-human red cell antibodies in mice following immunization with the liposomal formulations, sera (1:10 dilution) were collected four weeks after the third immunization. Mouse sera were incubated with batch matched human red blood cells (0.2% haematocrit) used in formulating the liposomes, for 30 minutes at room temperature. 50 μL of goat anti-mouse IgG AlexaFluor-488 (1:500 dilution) (Invitrogen) was added and the cells incubated for an additional 30 minutes. The cells were washed twice between each step with 1% FCS/PBS. At least 50,000 events were then acquired on a BD LSR Fortessa (BD BioSciences) and data analysed using FlowJo software version 10.0 (Tree Star).

### Example 3

### Assessment of Anti-Parasite Immune Responses

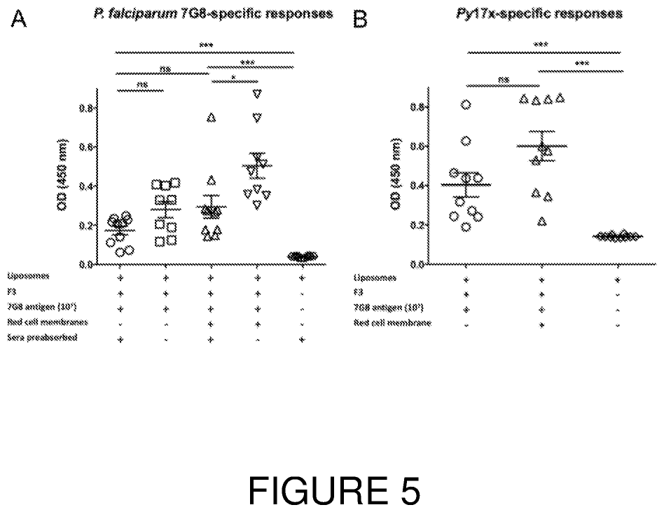

Although the induction of antibodies to P. falciparum antigens was initially measured, the induction of anti-hRBC antibodies (as illustrated in FIG. 4) can also contribute to the total antibody response. Therefore, to examine P. falciparum-specific antibody responses, anti-hRBC antibodies were pre-absorbed from the sera of liposome-vaccinated mice using the same batch of hRBCs that was used to culture the parasites contained in the vaccine formulation. Pre-absorption of sera from mice vaccinated with liposomes containing red cell membranes significantly reduced the antibody response to crude P. falciparum pRBC antigen, demonstrating that pre-absorption can remove red cell-specific antibodies (see, FIG. 5A). There was a residual antibody response in pre-absorbed sera from mice that received liposomes containing red cell membrane-depleted parasite antigens and this was significantly greater than the antibody response seen in sera from mice vaccinated with empty liposomes (P<0.001) (FIG. 5A). Although the amount of parasite-specific antibody was only modest, the level was consistent with the antibody responses following vaccination with other WP blood-stage vaccines (see, Raja et al., 2016; and Low et al., 2019).

The antibody response to a heterologous rodent parasite, P. yoelii 17X, was also examined, which demonstrated the induction of parasite-specific IgG (see, FIG. 5B).

### Materials and Methods

Preparation of Pre-Adsorbed Sera for ELISA

Sera (1:10 dilution) from liposome-immunized mice were pre-adsorbed to deplete anti-human red blood cell antibodies. An equal volume (30 μL) of sera and batch-matched human normal red blood cells (nRBCs) were added to an Eppendorf tube, incubated for 2 hours at 37° C., and then overnight at 4° C. The suspension was centrifuged at 10,000 g for 10 minutes to pellet the human nRBCs and the supernatant was used for ELISA.

Detection of Serum Antibodies by ELISA

For detection of antibodies in vaccinated mice, 96-well flat-bottomed immunoplates (MaxiSorp Nunc) were coated with 10 μg/mL of Py17X crude antigen or 5 μg/ml P. falciparum 7G8 crude antigen in bicarbonate coating buffer (pH 9.6) overnight at 4° C. Plates were subsequently blocked with blocking buffer (10% skim milk in PBS/0.05% Tween-20 (Chem-supply) for 2 hours at room temperature. Wells were washed twice (0.05% Tween-20 in PBS), 100 μL of serially diluted serum was dispensed into each well and incubated for 2 hours at 37° C. The plates were then washed 5 times and biotinylated anti-mouse-IgG (Invitrogen) (diluted 1:3000 in blocking buffer) was added and incubated for 1 hour at room temperature in the dark. Wells were washed five times to remove unbound antibodies, tetramethylbenzidine (TMB) (OptEIA™ BD Biosciences) was added and plates incubated at room temperature for 30 minutes. 1 M sulphuric acid was added to each well to stop the reaction. Absorbance was determined at 450 nm using a xMark™ (BIO-RAD) microplate spectrophotometer and parasite-specific antibody titres were calculated as three standard deviations above the mean of duplicate naïve control sera.

### Example 4

### Assessment of Cell-Mediated Immune Responses

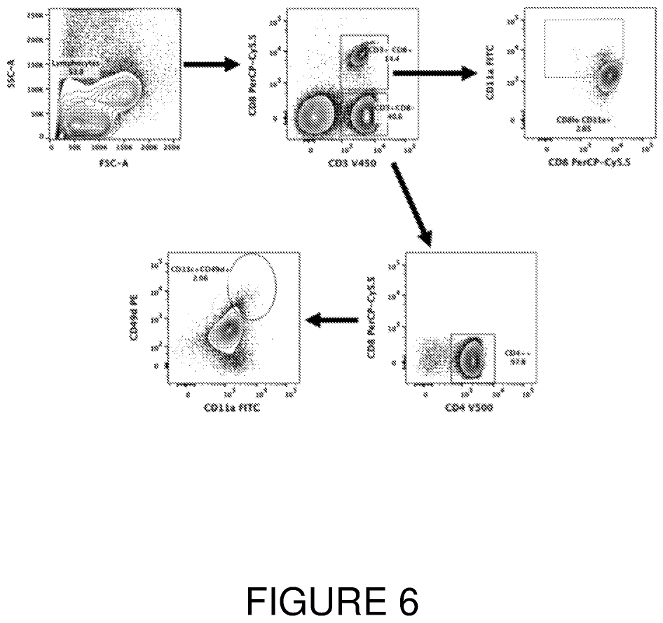

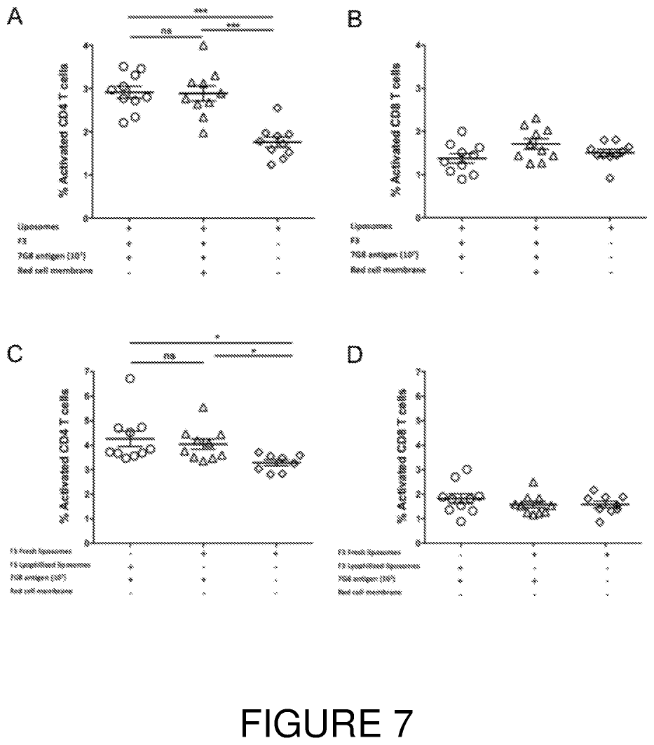

BALB/c mice were vaccinated with liposomes containing parasite antigen with or without removal of red cell membranes. In the first set of experiments, antigenic material from Preparation 1 (Table 1) was used to make the liposomes that had red cell membranes removed. Blood samples were collected for assessment of antigen-experienced T cells using early activation markers (CD49d^{hi}CD11a^{hi} for CD4⁺ and CD8^{lo}CD11a^{hi} for CD8⁺) seen days after the third immunization. FIG. 6 illustrates the gating strategy used to examine antigen-experienced T cells. An increase in the frequency of activated peripheral blood CD4⁺ T cells was observed in mice immunized with liposomes containing parasite antigen with or without depletion of red cell membranes (FIG. 7A) (P<0.001). There was no significant CD8⁺ T cell activation observed in these mice (FIG. 7B).

The study was repeated and the immunogenicity of fresh versus lyophilized liposomes was compared. Mice were only vaccinated with liposomes from which the red cell membranes were removed (Preparation 2, Table 2). Again, there was a significant increase in the number of activated CD4⁺ T cells, but not CD8⁺ T cells, in mice immunized with fresh liposomes and similar responses were observed in mice immunized with lyophilized liposomes (see, FIG. 7C, 7D).

### Materials and Methods

Formulation of Lyophilised Liposomes

For the preparation of lyophilized liposomes, hydration was performed using PBS 20 mM (pH 7.2-7.4) containing 10% trehalose (w/w) and parasite antigen. Thy hydrated liposomes were aliquoted into scintillation glass vials and snap-frozen on dry ice then dissolved in acetone for 10 minutes. With the caps loosened, the frozen vials were transferred to a 500 mL freeze dryer jar which was then connected to the freeze-dryer at −40° C. and 0.1 millibar vacuum for 18-20 hours. Following freeze-drying, the vials were tightly capped, sealed with parafilm and stored at −4° C. until required for immunization. Prior to immunization, lyophilized liposomes were rehydrated in 1× Dulbecco's PBS (DPBS). The average particle size in micrometres (μm) and size distribution of liposomes were measured using the Mastersizer 2000 (Malvern Instruments, England, UK).

Mice were immunized subcutaneously (s.c.) on day 0, 14, and 28 with freshly prepared or lyophilized liposomes in a volume of 200 μL in PBS. The vaccine does per mouse contained a parasite equivalent of approximately 1×10⁷. The parasite extract was either depleted or not depleted of red cell membranes. Each liposome dose administered per mouse contained 500 μg of DPPC, 200 μg of DDAB, 100 μg of cholesterol and 10 μg of 200 μl in PBS.

### Example 5

### Assessment of Spleen Cell Proliferation and Soluble Cytokine Responses

Approximately four weeks after the third immunization, mice from each group were sacrificed for spleen excision and assessment of parasite-specific proliferative responses and cytokine production. The induction of strain- and species-transcending cell-mediated immune responses to lysates from P. yoelii 17X, P. knowlesi, P. falciparum 7G8, and the clinical P. falciparum isolate UGMCB-009 were investigated.

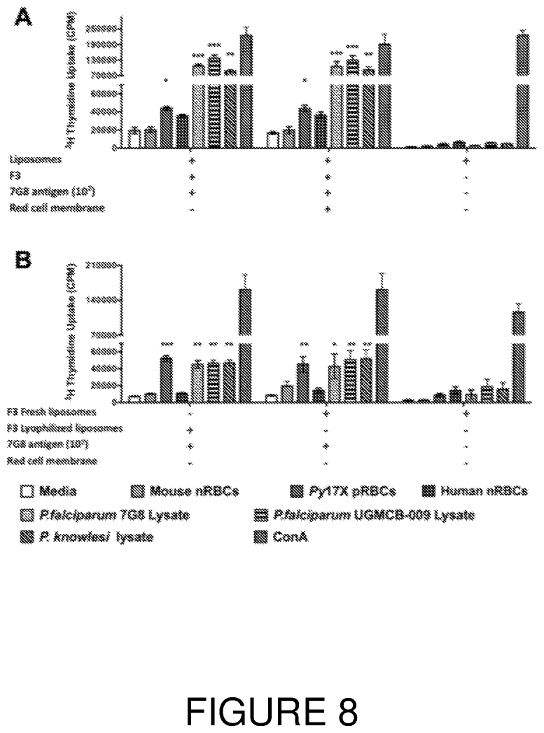

Immunization with liposomes containing parasite antigen with or without depletion of red cell membranes resulted in elevated splenocyte proliferative responses against all parasite antigen stimulants compared to mouse normal red blood cells (nRBCs) and human nRBCs (P<0.002) (see, FIG. 8A). No significant differences in parasite-specific proliferative responses were observed when comparing responses between mice immunized with liposomes containing red cell membrane-depleted antigens and those immunized with liposomes containing parasite antigens without red cell membrane depletion (FIG. 8A). Similarly, immunization with lyophilized liposomes resulted in significantly enhanced species- and strain-transcending splenocyte proliferative responses (P>0.033) (FIG. 8B). compared to the corresponding nRBC controls. There was no significant difference in proliferative responses between mice immunized with freshly prepared liposomes compared to lyophilized liposomes (see, FIG. 7B).

Supernatants obtained during splenocyte culture were then used for soluble cytokine analysis. Significant upregulation of IL-2, IFN-γ, TNF, IL-4, IL-6, and IL-10 was observed in mice immunized with liposomes containing parasite antigen with or without depletion of red cell membranes (P<0.033) with the only exception being P. knowlesi-specific TNF responses amongst mice immunized with liposomes containing parasite antigen not depleted of red cell membranes, where the response did not reach statistical significance (see, Table 3). Table 3 shows Th1 and Th2 cytokine responses in BALB/c mice immunized with freshly prepared F3 liposomes containing P. falciparum 7G8 antigens with or without red cell membranes. Spleen cells were incubated with Py17X parasitized red blood cells (pRBCs), P. falciparum 7G8 or UGMCB-009 lysate, P. knowlesi lysate, mouse normal red blood cells (nRBCs) and human nRBCs. Culture supernatants were collected for assessment of IL-2, IFN-γ, TNF, IL-4, IL-6, and IL-10 cytokine responses. The mean cytokine concentration (pg/ml) was expressed as ±standard error of the mean (SEM) (n=3 mice per group). Data were analysed using unpaired t test to compare Py17X-/P. falciparum 7G8-/UGMCB-009-/P. knowlesi-specific cytokine responses to mouse nRBC/human nRBC responses for each liposome formulation (*P<0.033, **P<0.002, ***P<0.001).

Immunization with lyophilized liposomes containing red cell membrane-depleted antigens resulted in similar Th1 cytokine responses to mice immunized with fresh liposomes, although the TNF response did not reach significance (see, Table 3). However, the Th2 responses were mostly reduced following immunization with lyophilized liposomes, compared to the responses observed following immunization with fresh liposomes (see, Table 4).

Table 4 presents the Th1 and Th2 cytokine responses in BALB/c mice immunized with fresh or lyophilized F3 liposomes containing red cell membrane depleted P. falciparum 7G8 antigens. Spleen cells were incubated with Py17X parasitized red blood cells (pRBCs), P. falciparum 7G8 or UGMCB-009 lysate, P. knowlesi lysate, mouse normal red blood cells (nRBCs) and human nRBCs. Culture supernatants were collected for assessment of IL-2, IFN-γ, TNF, IL-4, IL-6, and IL-10 cytokine responses. The mean cytokine concentration (pg/ml) was expressed as ±standard error of the mean (SEM) (n=3 mice per group). Data were analysed using unpaired t test to compare Py17X-/P. falciparum 7G8-/UGMCB-009-/P. knowlesi-specific cytokine responses to mouse nRBC/human nRBC responses for each liposome formulation (*P<0.033, **P<0.002, ***P<0.001).

Together, these data show that depletion of red cell membranes from parasite antigen does not affect vaccine immunogenicity with respect to the cytokine response. However, the lyophilized caving formulation induced a parasite-specific response that was skewed towards production of Th1 cytokines.

TABLE 3

| | Cytokine concentration (pg/ml) | | | | | | | | | | | |
|---|---|---|---|---|---|---|---|---|---|---|---|---|
| | F3 liposomes containing P. falciparum 7G8 antigens | | | | | | F3 liposomes containing P. falciparum 7G8 antigens | | | | | |
| | with red cell membrane depletion | | | | | | without red cell membrane depletion | | | | | |
| | | | | | UGMCB- | P. | | | | | UGMCB- | P. |
| | Mouse | Py17X | Human | 7G8 | 009 | knowlesi | Mouse | Py17X | Human | 7G8 | 009 | knowlesi |
| Cytokine | nRBCs | pRBCs | nRBCs | lysate | lysate | lysate | nRBCs | pRBCs | nRBCs | pRBCs | lysate | lysate |
| IL-2 | 61.17 ± | 179.17 ± | 102.75 ± | 459.60 ± | 362.23 ± | 390.83 ± | 82.02 ± | 164.33 ± | 91.32 ± | 386.06 ± | 370.90 ± | 271.78 ± |
| | 11.35 | 14.97 | 1.99 | 57.27 | 41.14 | 67.62 | 0.89 | 29.30 | 4.58 | 73.83 | 74.56 | 36.69 |
| | | ** | | *** | ** | ** | | * | | *** | ** | * |
| IFNγ | 14.14 ± | 196.35 ± | 111.53 ± | 1817.80 ± | 2919.39 ± | 1885.60 ± | 15.17 ± | 121.23 ± | 60.93 ± | 1563.71 ± | 2829.64 ± | 1723.95 ± |
| | 4.73 | 47.59 | 12.34 | 550.12 | 467.50 | 820.43 | 4.92 | 13.71 | 2.99 | 580.43 | 379.67 | 252.00 |
| | | ** | | *** | *** | ** | | * | | ** | *** | * |
| TNF | 3.84 ± | 28.23 ± | 27.98 ± | 109.39 ± | 75.30 ± | 87.37 ± | 2.72 ± | 28.91 ± | 24.94 ± | 122.34 ± | 104.06 ± | 40.74 ± |
| | 0.69 | 4.76 | 2.70 | 15.40 | 38.14 | 35.01 | 0.62 | 7.06 | 0.95 | 33.25 | 16.00 | 5.68 |
| | | ** | | ** | * | * | | ** | | ** | ** | |
| IL-4 | 6.09 ± | 40.55 ± | 25.46 ± | 57.06 ± | 180.94 ± | 145.24 ± | 5.18 ± | 25.00 ± | 50.87 ± | 119.86 ± | 180.16 ± | 101.20 ± |
| | 2.26 | 12.55 | 6.02 | 8.21 | 16.45 | 33.37 | 2.33 | 3.69 | 5.27 | 24.01 | 27.09 | 11.42 |
| | | ** | | * | *** | *** | | * | | ** | *** | * |
| IL-6 | 47.02 ± | 162.60 ± | 139.30 ± | 379.34 ± | 1227.39 ± | 406.19 ± | 54.51 ± | 366.24 ± | 162.91 ± | 540.25 ± | 1010.90 ± | 565.68 ± |
| | 12.56 | 18.13 | 20.62 | 53.07 | 229.22 | 69.13 | 6.61 | 83.43 | 21.77 | 190.34 | 143.39 | 103.89 |
| | | * | | * | *** | * | | *** | | * | *** | * |
| IL-10 | 3.71 ± | 49.04 ± | 81.33 ± | 448.24 ± | 609.76 ± | 520.12 ± | 12.04 ± | 41.42 ± | 108.66 ± | 446.47 | 630.58 ± | 500.42 ± |
| | 3.71 | 17.8 | 16.72 | 101.61 | 98.17 | 84.39 | 1.63 | 7.75 | 5.82 | ± 41.58 | 105.48 | 82.39 |
| | | ** | | *** | *** | *** | | * | | *** | *** | *** |

TABLE 4

| | Cytokine concentration (pg/ml) | | | | | | | | | | | |
|---|---|---|---|---|---|---|---|---|---|---|---|---|
| | Lyophilized F3 liposomes | | | | | | Freshly prepared F3 liposomes | | | | | |
| | | | | | UGMCB- | P. | | | | | UGMCB- | P. |
| | Mouse | Py17X | Human | 7G8 | 009 | knowlesi | Mouse | Py17X | Human | 7G8 | 009 | knowlesi |
| Cytokine | nRBCs | pRBCs | nRBCs | pRBCs | lysate | lysate | nRBCs | pRBCs | nRBCs | pRBCs | lysate | lysate |
| IL-2 | 10.60 ± | 29.24 ± | 81.35 ± | 301.07 ± | 553.54 ± | 431.86 ± | 20.37 ± | 173.51 ± | 126.03 ± | 491.69 ± | 659.43 ± | 625.33 ± |
| | 9.29 | 11.57 | 13.12 | 44.85 | 131.97 | 78.69 | 8.19 | 44.88 | 15.08 | 95.20 | 106.50 | 153.86 |
| | | | | *** | *** | *** | | ** | | ** | *** | *** |
| IFNγ | 0.43 ± | 102.92 ± | 137.37 ± | 1227.83 ± | 1123.13 ± | 748.67 ± | 0.93 ± | 170.63 ± | 99.54 ± | 955.14 ± | 1392.93 ± | 1265.84 ± |
| | 0.43 | 47.79 | 12.79 | 206.56 | 416.04 | 511.17 | 0.93 | 62.09 | 4.97 | 367.09 | 359.27 | 252.76 |
| | | *** | | *** | ** | * | | *** | | ** | ** | * |
| TNF | 1.08 ± | 1.40 ± | 12.82 ± | 14.13 ± | 35.52 ± | 24.15 ± | 0.00 ± | 3.98 ± | 8.73 ± | 46.05 ± | 76.55 ± | 46.23 ± |
| | 1.08 | 0.72 | 1.43 | 6.42 | 9.70 | 9.03 | 0.00 | 2.16 | 6.27 | 13.99 | 27.80 | 13.08 |
| | | | | | | | | | | | ** | |
| IL-4 | 1.03 ± | 3.36 ± | 5.38 ± | 13.78 ± | 36.72 ± | 39.88 ± | 0.83 ± | 10.52 ± | 5.79 ± | 25.69 ± | 63.71 ± | 38.73 ± |
| | 0.61 | 0.20 | 0.83 | 6.75 | 16.64 | 14.65 | 0.83 | 2.36 | 3.44 | 3.59 | 14.78 | 14.14 |
| | | | | | * | * | | * | | ** | ** | * |
| IL-6 | 57.19 ± | 93.63 ± | 117.06 ± | 228.69 ± | 336.34 ± | 280.63 ± | 47.87 ± | 273.30 ± | 86.65 ± | 363.94 ± | 594.42 ± | 607.39 ± |
| | 11.67 | 8.54 | 18.73 | 58.60 | 101.72 | 77.28 | 28.57 | 46.25 | 41.45 | 154.98 | 232.44 | 232.31 |
| | | | | | | | | *** | | * | ** | ** |
| IL-10 | 0.94 ± | 1.94 ± | 7.29 ± | 6.42 ± | 18.94 ± | 6.79 ± | 3.20 ± | 15.24 ± | 28.80 ± | 87.93 ± | 105.26 ± | 81.44 ± |
| | 0.67 | 0.15 | 1.59 | 2.83 | 2.39 | 2.13 | 0.27 | 2.59 | 11.59 | 10.99 | 31.75 | 27.18 |
| | | | | | | | | * | | ** | ** | ** |

### Materials and Methods

Assessment of Splenocyte Proliferation and Cytokine Production

Spleens were excised from mice into complete RPMI medium (RPMI 1640, supplemented with 1% L-glutamine, 10% foetal bovine serum, 0.1% 2-mercaptoethanol and 1% penicillin-streptomycin) and filtered through a 70 μm cell strainer using a sterile syringe plunger. The harvested spleen cells were washed in complete RPMI medium and the red blood cells were lysed using Gey's lysis buffer. Following lysis, spleen cells were washed with complete RPMI medium at 400 g for 5 minutes and resuspended at 5×10⁶ cells/mL in complete medium. Spleen cells were subsequently seeded into 96-well U-bottomed plates (5×10⁵ cells/well) and cultured for 72 hours at 37° C. and 5% CO₂ in the presence of complete medium (negative control), naïve mouse red blood cells (5×10⁶ nRBC/mL) (nRBCs, negative control), live Py17X pRBCs (5×10⁶ pRBC/mL) obtained from a Py17X infected mouse, normal human red blood cell lysate (5×10⁶ nRBC/mL), purified P. falciparum 7G8, P. falciparum UGMCB-009, P. knowlesi pRBC lysates (5×10⁶ pRBC/mL) or concanavalin A (10 μg/mL) (Con A, positive control) (Sigma-Aldrich) in triplicate. After 54 hours, culture supernatants were harvested for cytokine analysis. To assess proliferation, splenocytes were pulsed with 1 μCi of 3[H]-thymidine/well (Perkin Elmer) and cultured for an additional 18 hr. Plates were then frozen at −80° C., and later thawed for harvesting onto 1450 MicroBeta glass fibre filters (Wallac, USA). The filters were air dried and ³[H]-thymidine incorporation measured using a Microbeta2 Microplate Counter (Perkin Elmer) to obtain radioactivity counts per minute (CPM) values.

Soluble cytokine analysis was undertaken on the culture supernatants and was performed using the Th1/Th2/Th17 CBA kit (BD Biosciences) with a slight modification of the manufacturer's instructions as described previously (see, Raja et al., 2016). 10 μL of culture supernatants from pooled triplicate wells were incubated with a master mix containing 2 μL of each capture bead and an equal volume of PE detection reagent in a 96 well V-bottom plate (Sarstedt) for 2 hours at room temperature in the dark. Plates were washed by centrifugation at 800 g for 5 minutes, resuspended in wash buffer and transferred into FACS tubes for acquisition of on a BD LSR Fortessa flow cytometer (BD Biosciences). The acquired data was analysed using FCAP Array software version 1.0.1 (BD Biosciences).

Statistical Data Analysis

All statistical analyses were conducted using GraphPad Prism software version 6 (GraphPad Software, Inc., CA). All the data were expressed as arithmetic mean±standard error of the mean (SEM) unless stated otherwise. Data were analysed using unpaired Mann-Whitney t test or unpaired t test to compare study groups and controls. A P value of <0.05 was considered significant for all statistical analysis.

### Example 6

### Assessment of Soluble Parasite Antigen Extract

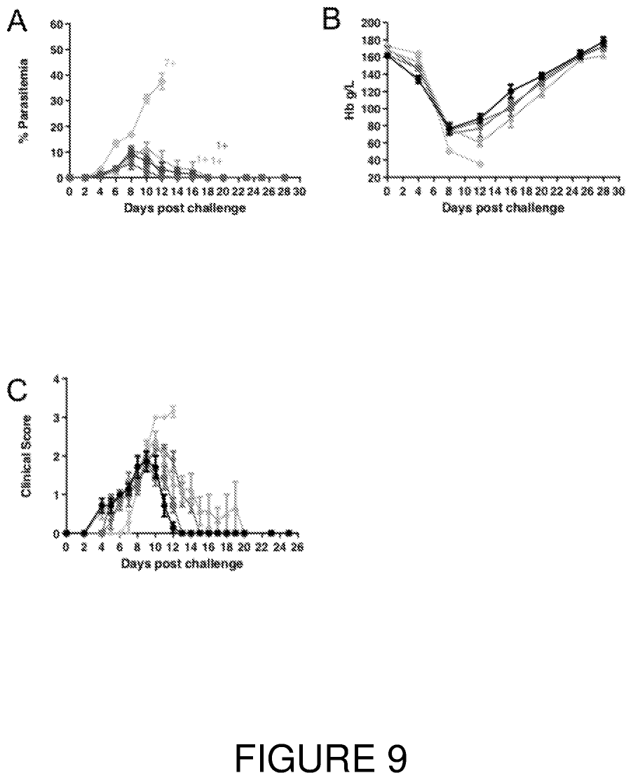

The above results led to the further investigation of the effectiveness of using soluble parasite extract in stimulating an immune response against parasite challenge. Mice were vaccinated with three doses of different amounts of P. yoelii 17X soluble asexual blood-stage parasite antigen encapsulated in liposomes. The liposomes presented the F3 cell-targeting ligand and PHAD molecules on their surfaces. Comparison studies with whole parasite lysate encapsulated in F3-PHAD liposomes were also performed, and parasitemia, haemoglobin, and clinical scores of the vaccinated mice were determined, after challenge with 1×10⁵ P. yoelii pRBC (see, FIG. 9).

Spleens were extracted from mice into complete RPMI medium (RPMI 1640, supplemented with 1% L-glutamine, 10% foetal bovine serum, 0.1% 2-mercaptoethanol and 1% penicillin-streptomycin) and filtered through a 70 μM cell strainer using a sterile syringe plunger. The harvested spleen cells were washed in complete RPMI medium and the RBCs were lysed using Gey's lysis buffer. Following lysis, spleen cells were washed with complete RPMI medium at 400 g for 5 min and resuspended at 5×10⁶ cells/mL in complete medium. Spleen cells were subsequently seeded into 96-well U-bottomed plates (5×10⁵ cells/well) and cultured for 72 hrs at 37° C. and 5% CO₂ in the presence of complete medium (negative control), naïve mouse RBCs (5×10⁶ nRBC/mL) (nRBCs, negative control), soluble Py17X extract derived from parasitized red blood cells (Py pRBCs) derived from mice vaccinated with different amounts of soluble antigen extract, or concanavalin A (10 μg/mL) (Con A, positive control) (Sigma-Aldrich) in triplicate. After 54 hrs, culture supernatants were harvested for cytokine analysis. To assess proliferation, splenocytes were pulsed with 1 μCi of 3[H]-thymidine (PerkinElmer)/well and cultured for an additional 18 hrs. Plates were then frozen at −80° C., and later thawed for harvesting onto 1450 MicroBeta glass fibre filters (Wallac, USA). The filters were air dried and ³[H]-thymidine incorporation measured using a Microbeta2 Microplate Counter (Perkin Elmer) to obtain radioactivity corrected counts per minute (CCPM) values.

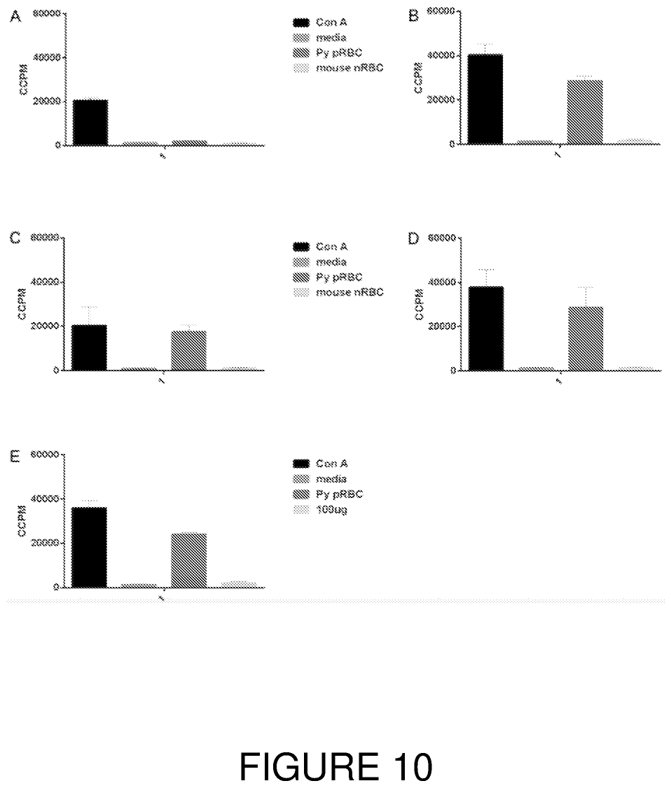

Approximately four weeks after the third immunisation at the indicated dose, mice from each group were sacrificed for spleen extraction and assessment of proliferative responses. Following in vitro stimulation, significant Py17X-specific splenocyte proliferative responses were observed in mice immunised with F3 liposomes containing RBC component-depleted soluble parasite extract (equivalent to 10⁶ and 10⁷ pRBC) compared to corresponding mouse nRBCs responses for each group (P<0.033) (see, FIG. 10). However, spleen cells extracted from mice immunised with media or naïve RBCs did not proliferate in response to Py17X (see, FIG. 10).

This data shows that the soluble parasite extract can induce proliferative responses comparable to the whole parasite lysate.

### Materials and Methods

Preparation of Soluble Parasite Extract

Parasitised red blood cells were harvested has described in previous examples, before being lysed. For P. falciparum preparations, the cells were incubated with 0.15%. chilled saponin in PBS, before being incubated at 37° C. for 20 minutes. For Py17X, 0.06% saponin was used, and cells were incubated at RT for 10 minutes. After lysis had occurred, the insoluble fraction was washed twice with 50 mL of 1×PBS, and centrifuged at 1,800 rpm for 10 minutes, dec=1. The wash step was repeated until the supernatant was no longer red in colour and had instead turned clear.

Supernatant was removed and a smear taken to ensure parasites are liberated from the RBCs. Pellet was frozen at −20° C. for at least 1 hour, and thawed at RT for 30 minutes. Parasite cell lysis continued to occur with three cycles of freezing at −80° C. for at least 2 hours and thawing at RT for 30 mins. The cell pellet is then disrupted with sonicator, twice 30 secs on, 30 secs off (on ice), on maximum intensity.

Pellets were then centrifuged at 12,000 rpm for 30 minutes with slow braking, to separate insoluble material from soluble parasite antigens. Immuno-affinity depletion of RBC membranes was then performed as described for previous examples.

Supernatant containing soluble parasite antigen transferred to clean sterile tube, without disrupting pellet.

### Example 7

### Assessment of Cross-Species Protection

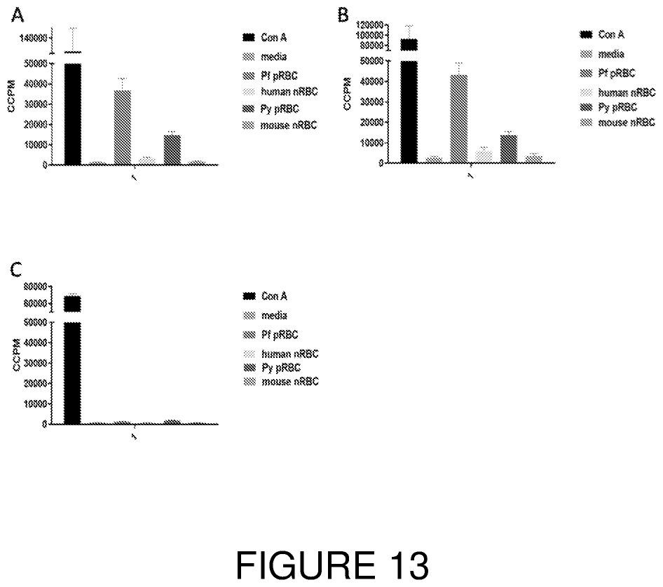

The inventors then investigated whether the soluble parasite extracts exhibit any cross-species parasite protection. FIG. 13 shows parasitemia data, haemoglobin data and clinical scores in mice that were vaccinated with 3 doses of: empty liposomes, P. falciparum soluble asexual blood-stage parasite antigen formulated with liposomes containing the F3 mannosylated core peptide and PHAD; or P. falciparum whole asexual blood-stage parasite antigen formulated with liposomes containing the F3 mannosylated core peptide and PHAD. Mice were challenged with 1×10⁵ P. yoelii pRBC.

Similarly, FIG. 13 shows P. falciparum and P. yoelii-specific splenocyte proliferative responses in mice immunised with 3 doses of (A) P. falciparum soluble asexual blood-stage parasite antigen formulated with liposomes containing the F3 mannosylated core peptide and PHAD; and (B) P. falciparum whole asexual blood-stage parasite antigen formulated with liposomes containing the F3 mannosylated core peptide and PHAD (C) empty liposomes.

This data shows that cross-species protection is provided by immunisation with the soluble parasite extracts of the invention. In other words, the soluble parasite extract compositions of the present invention are sufficient to provide homologous and heterologous protection against related parasites (e.g., P. falciparum and P. yoelii).

### Example 8

### Evaluation of the Induction of Anti-Human Red Blood Cell Antibodies in Mice

Construction of Liposomes and Removal of Red Cell Membranes

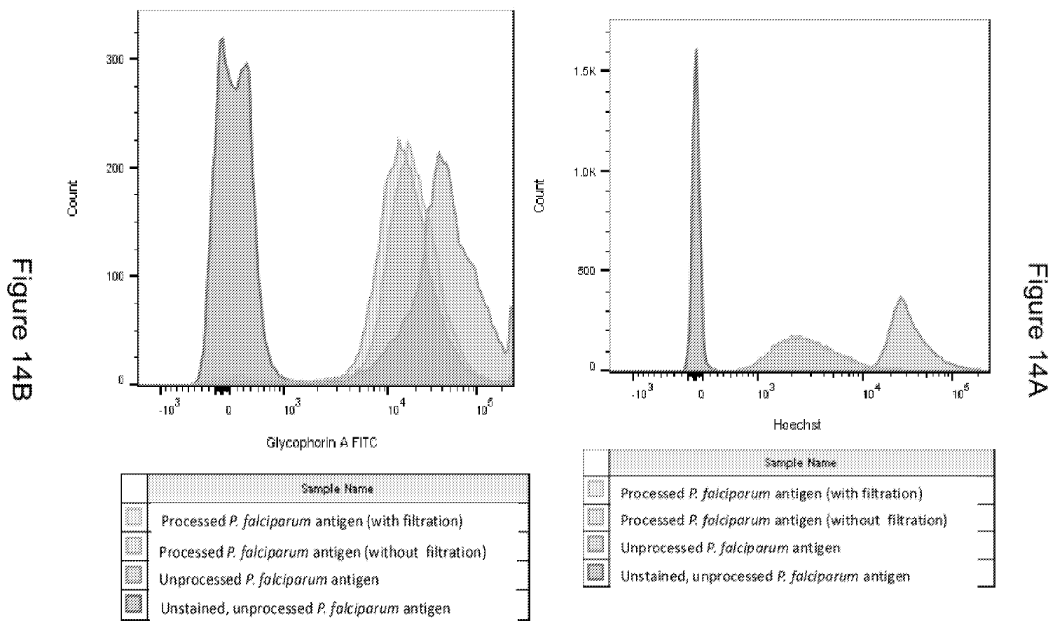

Liposomes were also constructed using parasite antigen from which human red cell membranes were removed using a combination of chemical lysis and centrifugation, with or without filtration to produce a soluble parasite extract. In the experiments described below, we used FACS to determine the median fluorescent intensity (MFI) of soluble parasite antigen following staining with antibodies to GlyA (to determine the presence of red cell membranes) and Hoechst to measure parasite DNA (as a surrogate for parasite antigen) (see, FIG. 14).

After establishing the methodology for preparation of soluble parasite extract using chemical lysis and centrifugation, two preparations were made (with and without subsequent filtration). It was estimated, based on Glycophorin A staining, that there was 66% and 60% depletion of red cell membranes in preparations 1 (without filtration) and 2 (with filtration), respectively compared with the unprocessed purified trophozoites (FIG. 14).

The amount of DNA remaining after preparing the soluble parasite extract, based on Hoechst staining, was similar between the two preparations with a depletion of 88% and 89% for preparations 1 and 2 respectively. Unlike the procedure followed for the immunomagnetic beads, this was not used to calculate the immunizing dose for the mice. Rather, the amount of soluble antigen preparation administered per vaccine dose was calculated based on the number of pRBCs that were used as the starting material. Thus, each vaccine dose contained soluble antigen from 10⁷ parasite equivalents (FIG. 14).

Assessment of Anti-Human RBC Antibodies

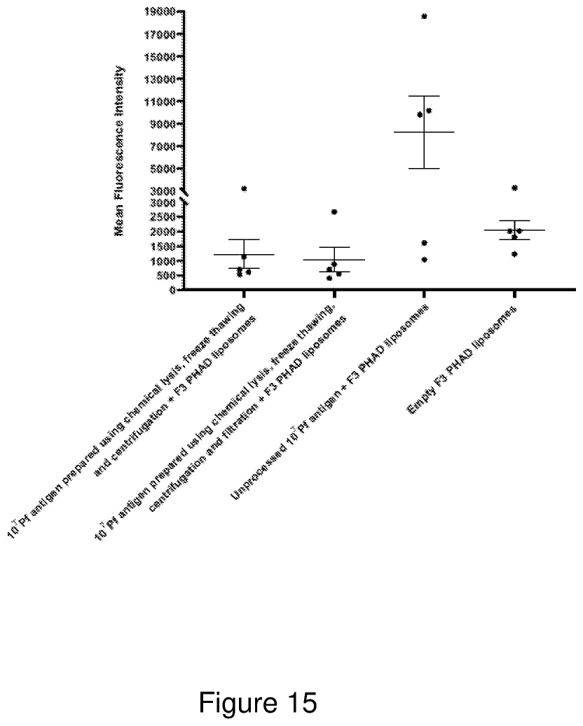

It was then assessed whether immunization of mice with F3/PHAD liposomes containing red cell membrane-depleted P. falciparum antigens resulted in the induction of anti-human red blood cell (hRBC) antibodies. For these experiments, parasite antigen from preparation 1 (chemical lysis and centrifugation without filtration) and preparation 2 (chemical lysis and centrifugation with filtration) was used to construct the liposomes from which red cell membranes were removed. A third preparation, where red blood cell membranes were not removed (unprocessed Pf antigen) was included as a comparative group. Mice immunized with empty liposomes (i.e., no parasite antigen) were also used as a control. Flow cytometric analyses revealed a significant reduction in anti-hRBC antibodies in mice immunized with P. falciparum soluble antigen preparations 1 (p=0.0278) and 2 (p=0.0159) compared to mice that received liposomes made with P. falciparum parasite antigen without depletion of red cell membranes (i.e., unprocessed pRBCs) (see, FIG. 15). These data support the use of soluble antigen preparations as a method to reduce the induction of alloantibodies to human red cells.

### Materials & Methods

Mice

Inbred Bagg Albino (BALB/c) (H-2d) female mice aged 4-6 weeks were purchased from the Animal Resource Centre (ARC) (Canning Vale, Western Australia). All animals were housed under special pathogen free, Physical Containment level 2 (PC2) conditions in the Griffith University Institute for Glycomics animal facility.

All animal procedures were performed in accordance with the Australian Code for the Care and Use of Animals for Scientific Purposes 8th edition (2013) and approved by Griffith University Animal Ethics Committee under ethics approval number GLY/06/20/AEC.

Human and Rodent Malaria Parasites

All liposomes were formulated using a P. falciparum 7G8 laboratory line (see, Stanisic et al., 2015).

Culture of P. falciparum Parasitized Red Blood Cells

Frozen vials containing P. falciparum parasitized red blood cells (pRBCs) in glycerolyte solution (Baxter) were thawed and cultured as described previously (see, Stanisic et al., 2015, Ssemaganda et al., 2018). Parasite cultures were monitored regularly by thin blood films. Thin blood films were stained with Giemsa and observed by microscopy to ascertain the parasitaemaia and life-cycle stage. Mature parasite forms (trophozoites and schizonts), purified from parasite cultures (as described below), were used for preparation of whole parasite antigen in liposomes (P. falciparum).

Purification of P. falciparum Trophozoites

To reduce the number of uninfected red blood cells, present in the parasite culture for the vaccine, trophozoite/schizont-stage pRBC were purified from culture by magnetic separation using CS columns (Miltenyi Biotec) on the MACS Vario system (Miltenyi Biotec). The pRBC preparations at >98% purity were treated as outlined below to facilitate removal of Red Cell Membranes.

Depletion of Red Cell Membranes From Purified P. falciparum Preparations

pRBC that were not undergoing any further processing (i.e., unprocessed), were pelleted and frozen at −80° C. until required. For the pRBC that were being further processed to deplete red cell membranes, the cells were incubated with 0.15% chilled saponin in PBS, before being incubated at 37° C. for 20 minutes. After lysis had occurred, the insoluble fraction was washed twice with 50 mL of 1×PBS, and centrifuged at 1,800 rpm for 10 minutes, dec=1. The wash step was repeated until the supernatant was no longer red in colour and had instead turned clear.

Supernatant was removed and a smear taken to ensure parasites are liberated from the RBCs. Pellet was frozen at −20° C. for at least 1 hour and thawed at RT for 30 minutes. Parasite cell lysis continued to occur with three cycles of freezing at −80° C. for at least 2 hours and thawing at RT for 30 mins. The cell pellet was then disrupted with sonicator, twice 30 secs on, 30 secs off (on ice), on maximum intensity.

Pellets were then centrifuged at 14,800 rpm for 30 minutes with slow braking, to separate insoluble material from soluble parasite antigens. Supernatant containing soluble parasite antigen was transferred to clean sterile tube, without disrupting pellet. For preparation 2, filtration of the sample was undertaken at this point with a low protein-binding 0.22 μM filter.

These parasite antigen extracts were used in formulating liposomes containing red-cell membrane depleted antigens. To confirm the removal of red cell membranes and the presence of parasite antigen following magnetic purification, 200 μL of the parasite extract was centrifuged for 10 min at 16,000 g. The pellet was subsequently stained with anti-Glycophorin A FITC (Clone H1264, BioLegend) and bisbenzimide Hoechst (Sigma-Aldrich) for 20 min at room temperature, then washed in MACS buffer. Samples were acquired on a BD LSR Fortessa (BD Biosciences) and data analysed using FlowJo software version 10.0 (Tree Star)(FIG. 14).

Preparation of P. falciparum Parasite Antigen without Depletion of Red Cell Membrane Antigen

Plasmodium falciparum-infected red cells at the desired concentration based on parasite equivalence prior to red cell membrane depletion were subjected to six freeze-thaw cycles and resuspended in an appropriate volume of PBS ready for use in the preparation of the liposomes.

Formulation of Liposomes Containing P. falciparum Antigens and Immunizations

Liposomes were prepared using the thin film hydration method as described previously with modifications (see, Giddam et al., 2016). Liposomes consisted of 1,2-dipalmitoylsn-glycero-3-phosphocholine (DPPC, Avanti polar lipids), dimethyldioctadecylammonium bromide (DDAB, Sigma-Aldrich) and cholesterol taken in the ratio of 7:2:1. Additionally, 10 μg of the mannosylated lipid core peptide, referred to as ‘F3’ hereon in, comprised of a lysine-serine-serine (KSS) spacer and 2-amino-D, L-hexadecanoic acid and 25 μg of 3D(6-acyl) PHAD (Avanti Polar Lipids) were added per dose of vaccine formulation. F3 was synthesized as previously described (see, Giddam et al., 2016). F3 was dissolved in methanol and all other components were dissolved in chloroform. The solvent mixture which was evaporated under vacuum to form a thin lipid film in the glass flask. The thin film was hydrated at 50-55° C. with parasite antigen (10⁷ P. falciparum parasite equivalents of soluble antigen per vaccine dose) in PBS or PBS alone (for empty liposomes).

Anti-Human Red Cell Antibody Binding Assay

To examine the induction of anti-human red cell antibodies in mice following immunization with the liposomal formulations, sera (1:10 dilution) were collected four weeks after the third immunization. Mouse sera were incubated with human red blood cells (0.2% haematocrit) used in formulating the liposomes, for 30 minutes at room temperature. 50 μL of goat anti-mouse IgG AlexaFluor-488 (1:500 dilution) (Invitrogen) was added and the cells incubated for an additional 30 minutes. The cells were washed twice between each step with 1% FCS/PBS. At least 50,000 events were then acquired on a BD LSR Fortessa (BD BioSciences) and data analysed using FlowJo software version 10.0 (Tree Star).

Statistical Data Analysis

All statistical analyses were conducted using GraphPad Prism software (GraphPad Software, Inc., CA). All the data were expressed as arithmetic mean±standard error of the mean (SEM) unless stated otherwise. Data were analysed using an unpaired Mann-Whitney t test to compare study groups and controls. A p value of <0.05 was considered significant for all statistical analysis.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### REFERENCES

Arnold, M. S., Engel, J. A., Chua, M. J., Fisher, G. M., Skinner-Adams, T. S., and Andrews, K. T. 2016. Adaptation of the [3H]Hypoxanthine Uptake Assay for in vitro-Cultured Plasmodium knowlesi Malaria Parasites. Antimicrob Agents Chemother 60 (7):4361-3.
Brain, M. C., Prevost, J. M., Pihl, C. E., and Brown, C. B. 2002. Glycophorin A-mediated haemolysis of normal human erythrocytes: evidence for antigen aggregation in the pathogenesis of immune haemolysis. Br J Haematol 118 (3):899-908.
Brito, L. A. and O'hagan, D. T. 2014. Designing and building the next generation of improved vaccine adjuvants. J Control Release 190:563-79.
Cavacini, L. A., Long, C. A., Weidanz, W. P., (1986) T-cell immunity in murine malaria: adoptive transfer of resistance to Plasmodium chabaudi adami in nude mice with splenic T cells, Infect Immun 52(3): 637-43
Coler, R. N., Carter, D., Friede, M., and Reed, S. G. 2009. Adjuvants for malaria vaccines. Parasite Immunol 31 (9):520-8.
Copland, M. J., Baird, M. A., Rades, T., Mckenzie, J. L., Becker, B., Reck, F., Tyler, P. C., and Davies, N. M. 2003. Liposomal delivery of antigen to human dendritic cells. Vaccine 21 (9-10):883-90.
Epstein, J. E., Paolino, K. M., Richie, T. L., Sedegah, M., Singer, A., Ruben, A. J., Chakravarty, S., Stafford, A., Ruck, R. C., Eappen, A. G., et al. 2017. Protection against Plasmodium falciparum malaria by PfSPZ Vaccine. JCI Insight 2 (1):e89154.
Ewer, K. J., O'hara, G. A., Duncan, C. J. A., Collins, K. A., Sheehy, S. H., Reyes-Sandoval, A., Goodman, A. L., Edwards, N.J., Elias, S. C., Halstead, F. D., et al. 2013. Protective CD8(+) T-cell immunity to human malaria induced by chimpanzee adenovirus-MVA immunisation. Nat Commun 4.
Genton, B., Betuela, I., Felger, I., Al-Yaman, F., Anders, R. F., Saul, A., Rare, L., Baisor, M., Lorry, K., Brown, G. V., et al. 2002. A recombinant blood-stage malaria vaccine reduces Plasmodium falciparum density and exerts selective pressure on parasite populations in a phase 1-2b trial in Papua New Guinea. J Infect Dis 185 (6):820-827.
Giddam, A. K., Reiman, J. M., Zaman, M., Skwarczynski, M., Toth, I., and Good, M. F. 2016. A semi-synthetic whole parasite vaccine designed to protect against blood stage malaria. Acta Biomater. DOI: 10.1016/j.actbio.2016.08.020.
Good, M. F., Reiman, J. M., Rodriguez, I. B., Ito, K., Yanow, S. K., El-Deeb, I. M., Batzloff, M. R., Stanisic, D. I., Engwerda, C., Spithill, T., et al. 2013. Cross-species malaria immunity induced by chemically attenuated parasites. J Clin Invest. 123 (8):3353-3362.
Ishizuka, A. S., Lyke, K. E., Dezure, A., Berry, A. A., Richie, T. L., Mendoza, F. H., Enama, M. E., Gordon, I. J., Chang, L. J., Samar, U. N., et al. 2016. Protection against malaria at 1 year and immune correlates following PfSPZ vaccination. Nat Med 22 (6):614-23.
Low, L. M., Ssemaganda, A., Liu, X. Q., Ho, M. F., Ozberk, V., Fink, J., Sundac, L., Alcorn, K., Morrison, A., O'callaghan, K., et al. 2019. Controlled Infection Immunization Using Delayed Death Drug Treatment Elicits Protective Immune Responses to Blood-Stage Malaria Parasites. Infect Immun 87 (1).
Lyke, K. E., Ishizuka, A. S., Berry, A. A., Chakravarty, S., Dezure, A., Enama, M. E., James, E. R., Billingsley, P. F., Gunasekera, A., Manoj, A., et al. 2017. Attenuated PfSPZ Vaccine induces strain-transcending T cells and durable protection against heterologous controlled human malaria infection. Proc Natl Acad Sci USA 114 (10):2711-2716.
Moon, R. W., Hall, J., Rangkuti, F., Ho, Y. S., Almond, N., Mitchell, G. H., Pain, A., Holder, A. A., and Blackman, M. J. 2013. Adaptation of the genetically tractable malaria pathogen Plasmodium knowlesi to continuous culture in human erythrocytes. Proc Natl Acad Sci USA 110 (2):531-6.
Mordmuller, B., Surat, G., Lagler, H., Chakravarty, S., Ishizuka, A. S., Lalremruata, A., Gmeiner, M., Campo, J. J., Esen, M., Ruben, A. J., et al. 2017. Sterile protection against human malaria by chemoattenuated PfSPZ vaccine. Nature 542 (7642):445-449.
Neafsey, D. E., Juraska, M., Bedford, T., Benkeser, D., Valim, C., Griggs, A., Lievens, M., Abdulla, S., Adjei, S., Agbenyega, T., et al. 2015. Genetic Diversity and Protective Efficacy of the RTS,S/AS01 Malaria Vaccine. N Engl J Med 373 (21):2025-2037.
Ogutu, B. R., Apollo, O. J., Mckinney, D., Okoth, W., Siangla, J., Dubovsky, F., Tucker, K., Waitumbi, J. N., Diggs, C., Wittes, J., et al. 2009. Blood Stage Malaria Vaccine Eliciting High Antigen-Specific Antibody Concentrations Confers No Protection to Young Children in Western Kenya. Plos One 4 (3).
Olotu, A., Fegan, G., Wambua, J., Nyangweso, G., Leach, A., Lievens, M., Kaslow, D. C., Njuguna, P., Marsh, K., and Bejon, P. 2016. Seven-Year Efficacy of RTS,S/AS01 Malaria Vaccine among Young African Children. N Engl J Med 374 (26):2519-29.
Perrie, Y., Crofts, F., Devitt, A., Griffiths, H. R., Kastner, E., and Nadella, V. 2016. Designing liposomal adjuvants for the next generation of vaccines. Adv Drug Deliv Rev 99 (Pt A):85-96.
Pinzon-Charry, A., Mcphun, V., Kienzle, V., Hirunpetcharat, C., Engwerda, C., Mccarthy, J., and Good, M. F. 2010. Low doses of killed parasite in CpG elicit vigorous CD4+ T cell responses against blood-stage malaria in mice. J Clin Invest 120 (8):2967-78.
Raja, A. I., Cai, Y., Reiman, J. M., Groves, P., Chakravarty, S., Mcphun, V., Doolan, D. L., Cockburn, I., Hoffman, S. L., Stanisic, D. I., et al. 2016. Chemically Attenuated Blood-Stage Plasmodium yoelii Parasites Induce Long-Lived and Strain-Transcending Protection. Infect. Immun. 84 (8):2274-88.
Raja, A. I., Stanisic, D. I., Good, M. F., 2017. Chemical Attenuation in the Development of a Whole-Organism Malaria Vaccine. Infect Immun. 85 (7):e00062-17.
Seder, Chang, L. J., Enama, M. E., Zephir, K. L., Samar, U. N., Gordon, I. J., Holman, L. A., James, E. R., Billingsley, P. F., Gunasekera, A., et al. 2013. Protection against malaria by intravenous immunization with a nonreplicating sporozoite vaccine (PfSPZ). Science 341 (6152):1359-65.
Sirima, S. B., Cousens, S., and Druilhe, P. 2011. Protection against malaria by MSP3 candidate vaccine. N Engl J Med 365 (11):1062-4.
Sissoko, M. S., Healy, S. A., Katile, A., Omaswa, F., Zaidi, I., Gabriel, E. E., Kamate, B., Samake, Y., Guindo, M. A., Dolo, A., et al. 2017. Safety and efficacy of PfSPZ Vaccine against Plasmodium falciparum via direct venous inoculation in healthy malaria-exposed adults in Mali: a randomised, double-blind phase 1 trial. Lancet Infect Dis 17 (5):498-509.
Ssemaganda, A., Low, L. M., Verhoeft, K. R., Wambuzi, M., Kawoozo, B., Nabasumba, S. B., Mpendo, J., Bagaya, B. S., Kiwanuka, N., Stanisic, D. I., et al. 2018. Gold(i) phosphine compounds as parasite attenuating agents for malaria vaccine and drug development. Metallomics 10 (3):444-454.
Ssemaganda, A., Giddam, A. K., Zaman, M., Skwarczynski, M., Toth, I., Stanisic, D. I., and Good, M. F. 2019. Induction of Plasmodium-specific immune responses using liposome-based vaccines.
Stanisic, D. I., Liu, X. Q., De, S. L., Batzloff, M. R., Forbes, T., Davis, C. B., Sekuloski, S., Chavchich, M., Chung, W., Trenholme, K., et al. 2015. Development of cultured Plasmodium falciparum blood-stage malaria cell banks for early phase in vivo clinical trial assessment of anti-malaria drugs and vaccines. Malar J. 14 (1):143.
Stanisic, D. I., Fink, J., Mayer, J., Coghill, S., Gore, L., Liu, X. Q., El-Deeb, I., Rodriguez, I. B., Powell, J., Willemsen, N. M., et al. 2018. Vaccination with chemically attenuated Plasmodium falciparum asexual blood-stage parasites induces parasite-specific cellular immune responses in malaria-naive volunteers: a pilot study. BMC Med 16 (1):184.
Su, Z., Tam, M. F., Jankovic, D., and Stevenson, M. M. 2003. Vaccination with novel immunostimulatory adjuvants against blood-stage malaria in mice. Infect Immun 71 (9):5178-87.
Ting, L. M., Gissot, M., Coppi, A., Sinnis, P., and Kim, K. 2008. Attenuated Plasmodium yoelii lacking purine nucleoside phosphorylase confer protective immunity. Nat Med 14 (9):954-8.
Thera, M. A., Doumbo, O. K., Coulibaly, D., Laurens, M. B., Ouattara, A., Kone, A. K., Guindo, A. B., Traore, K., Traore, I., Kouriba, B., et al. 2011. A field trial to assess a blood-stage malaria vaccine. N Engl J Med 365 (11):1004-13.
Vyas, S. P., Goyal, A. K., and Khatri, K. 2010. Mannosylated liposomes for targeted vaccines delivery. Methods Mol Biol 605:177-88.
Zaman, M., Ozberk, V., Langshaw, E. L., McPhun, V., Powell, J. L., Phillips, Z. N., et al., (2016). Novel platform technology for modular mucosal vaccine that protects against sterptococcus. Sci Rep, 6.

1. An immunostimulatory composition comprising a soluble parasite extract, wherein the composition is substantially free of insoluble parasite components or red blood cell (RBC) components.

2. The composition of claim 1, wherein the soluble parasite extract:
(a) is contained in or otherwise associated with a particle;
(b) comprises, consists, or consists essentially of substantially all the soluble parasite molecules present in the parasite; and/or
(c) is substantially free of detergent.

3.-6. (canceled)

7. The composition of claim 1, wherein the parasite:
(a) is an apicomplexan; and/or
(b) belongs to a genus selected from Plasmodium and Babesia.

8. (canceled)

9. The composition of claim 7, wherein
(a) the Plasmodium parasite is selected from the species Plasmodium falciparum, P. malariae, P. ovale, P. vivax, and P. knowlesi, or a combination thereof; or
(b) the Babesia parasite is selected from the species Babesia bigemina, B. bovis, B. caballi, B. canis, B. divergens, B. microti, and B. motasi, or a combination thereof.

10. (canceled)

11. The composition of claim 1, wherein the composition comprises two or more species of parasite from a single genus.

12. The composition of claim 2, wherein the particle is:
(a) a lipid vesicle;
(b) a liposome; and/or
(c) capable of being phagocytosed by an immune cell.

13.-14. (canceled)

15. The composition of claim 1, wherein the composition comprises a cell-targeting ligand, and optionally wherein the cell-targeting ligand targets the composition to an immune cell.

16. (canceled)

17. The composition of claim 15, wherein the immune cell is an antigen presenting cell (APC) selected from the group consisting of a dendritic cell and macrophage.

18. (canceled)

19. The composition of claim 15, wherein the cell-targeting ligand comprises:
(a) a lipid anchor component comprising:
(i) one or two lipid molecules; and/or
(ii) at least one palmitic acid molecule,
(b) a linker component optionally comprising:
(i) at least one amino acid residue; and/or
(ii) one or more polyethylene glycol (PEG) molecules, and
(c) an oligosaccharide component, optionally comprising at least one mannose residue.

20.-25. (canceled)

26. The composition of claim 15, wherein the cell-targeting ligand is F3 or F4.

27. The composition of claim 1, wherein the composition further comprises an adjuvant.

28. The composition of claim 27, wherein the adjuvant:
(a) is a TLR4 agonist; and/or
(b) is encapsulated within a particle, at least partially embedded within a particle, or located outside of a particle.

29. The composition of claim 28, wherein the TLR4 agonist is a Monophosphoryl Lipid A (MPLA) molecule, or a derivative thereof.

30. (canceled)

31. The composition of claim 29, wherein the particle is a liposome, and the adjuvant is at least partially embedded in the lipid bilayer of the liposome.

32. The composition of claim 1, wherein the particle is a liposome that comprises both a cell targeting ligand and an adjuvant, wherein the cell targeting ligand is F3 and the adjuvant is MPLA (PHAD®).

33. The composition of claim 31, wherein the particle comprises CAF01.

34. The composition of claim 1, wherein the composition is:
(a) formulated as a vaccine;
(b) cryopreserved or freeze dried;
(c) lyophilized; and/or
(d) rehydrated.

35.-37. (canceled)

38. A method of preparing an immunomodulatory composition for eliciting an immune response to a parasite antigen, the method comprising:
harvesting parasitized red blood cells (pRBCs);
lysing the pRBC cells under conditions sufficient to lyse the membrane of red blood cells but not sufficient to significantly lyse the parasite membranes;
harvesting the insoluble fraction of the pRBC lysate, wherein the insoluble fraction comprises red blood cell membranes and whole parasites;
lysing the parasite membranes; and
harvesting the soluble parasite fraction, wherein the soluble parasite fraction comprises soluble parasite antigens; to thereby produce an immunogenic composition sufficient to elicit an immune response to the parasite.

39.-48. (canceled)

49. A method of eliciting an immune response to a parasite antigen in a subject, the method comprising administering the composition of claim 1 comprising a soluble parasite extract contained in or otherwise associated with a particle, to thereby elicit an immune response in the subject.

50. A method of preventing or treating a parasitic disease in a subject, the method comprising administering the composition of claim 1 comprising a soluble parasite extract contained in or otherwise associated with a particle, to thereby prevent or treat the parasitic disease in the subject.

51.-88. (canceled)

87. An immunostimulatory composition comprising a whole parasite extract contained in or otherwise associated with a particle, wherein the whole parasite antigen component is substantially free or completely free of red blood cell components.

88. The method of claim 50, wherein the parasitic disease is malaria, and the soluble parasite extract being derived from a Plasmodium parasite.

89. The composition of claim 27, wherein the adjuvant is a lipid.
